(19) United States
(12) Patent Application Publication
(10) Pub. No.: US 2009/0099356 A1
(43) Pub. Date: Apr. 16, 2009

(54) PREPARATION OF 7-ALKENYL-3 QUINOLINECARBONITRILES VIA A PALLADIUM MEDIATED COUPLING REACTION

(75) Inventors: Yanong Daniel Wang, Warren, NJ (US); Dan Maarten Berger, New York, NY (US); Minu Dutia, West Nyack, NY (US); Middleton Brawner Floyd, Suffern, NY (US)

Correspondence Address: FITZPATRICK CELLA (WYETH), 30 ROCKEFELLER PLAZA, NEW YORK, NY, 10112-3800, US

(73) Assignee: WYETH, Madison, NJ (US)

(21) Appl. No.: 12/161,999

(22) PCT Filed: Jan. 23, 2007
(86) PCT No.: PCT/US07/01750

§ 371 Date: Dec. 11, 2008

Related U.S. Application Data
(60) Provisional application No. 60/771,903, filed on Feb. 8, 2006.

(51) Int. Cl. C07D 215/38; C07D 495/04; C07D 401/02; C07D 413/02

(52) U.S. Cl. 544/128; 546/159; 546/160; 546/114; 544/363

(57) ABSTRACT

The present invention is directed to a process for preparing compounds of formula (I): wherein A, R¹-R³, X, s, t, u, m and Z are defined herein, comprising the step of reacting a reagent of formula (II): in the presence of Pd(O) metal with a compound of formula (III): or salts thereof. Another aspect of this invention is a method of preparing compounds of formula (VI).

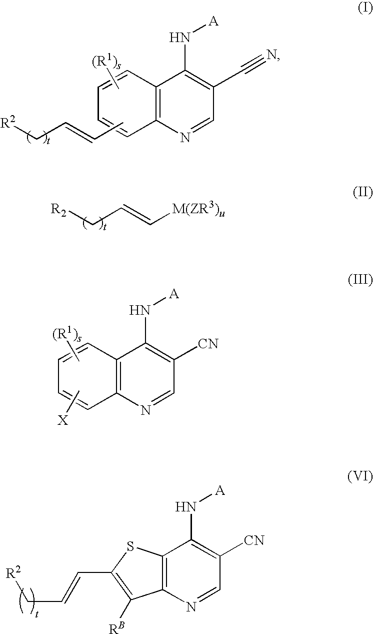

This application claims the benefit of U.S. Provisional Patent Application No. 60/771,903, filed Feb. 8, 2006, the disclosure of which is incorporated by reference herein.

### BACKGROUND OF THE INVENTION

1. Field of the Invention

This invention relates to a new synthetic approach for the preparation of 7-alkenyl-3-quinolinecarbonitriles and 2-alkenyl-5-thienopyridinecarbonitriles using a palladium mediated coupling reaction.

2. Related Background Art

The compounds synthesized by the method of the present invention are known to be inhibitors of protein kinases required for cell growth and differentiation. These compounds are useful for the treatment of certain diseases in mammals, for example cancers, osteoporosis and polycystic kidney disease. U.S. Pat. Nos. 6,521,618 and 6,689,772 disclose 3-cyanoquinoline compounds which exhibit such activity.

International Publication No. WO 2004/048286 discloses thieno[3,2-b]pyridine carbonitrile compounds which also possess protein kinase inhibitory activity useful in the treatment of cancers in mammals.

The prior references only disclose non-stereoselective methods of synthesizing these types of compounds. The present invention, however, involves synthesizing these compounds using a stereoselective palladium mediated coupling, which provides the desired E-isomer in excellent yields, and is therefore superior to prior disclosed methodology.

### BRIEF DESCRIPTION OF THE INVENTION

A process for preparing compounds of formula (I):

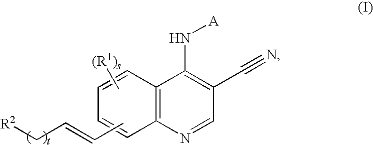

wherein R¹ is independently selected from H, alkyl of 1 to 6 carbon atoms, C₁-C₁₂ alkoxy, F, Cl and CF₃, R² is selected from the group H, alkyl of 1 to 6 carbon atoms, OH, Cl, F, acetyl, —OSO₂—C₆-C₁₂ aryl, —OSO₂—C₁-C₁₂ alkyl and —NR¹⁹R²⁰, where R¹⁹ and R²⁰ can independently be H and alkyl of 1 to 6 carbon atoms, or R¹⁹ and R²⁰ taken together form a 3 to 8 membered heterocycle containing 1-3 heteroatoms selected from O, S, and N, and where R¹⁹ and R²⁰ can be substituted with groups selected from C₁-C₆ alkylamino, C₂-C₁₂ dialkylamino, and a 3-8 membered heterocycle containing 1-3 heteroatoms selected from O, S, and N, A is aryl of 6 to 12 carbon atoms optionally substituted with 1 to 4 substituents which are independently selected from H, J, NO₂, NH₂, OH, SH, CN, COOH, CONH₂, NHC(O)NH₂, C(O)H, CF₃, OCF₃, R⁵, OR⁵, NHR⁵, Q, S(O)ₘR⁵, NHSO₂R⁵, R₆OH, R₆OR₅, R₆NH₂, R⁶NHR⁵, R⁶Q, R⁶SH, R⁶S(O)ₘR⁵, NHR⁷OH, NHR⁷OR⁵, N(R⁵)R⁷OH, N(R⁵)R⁷OR⁵, NHR⁷NH₂, NHR⁷NHR⁵, NHR⁷Q, N(R⁵)R⁷NH₂, N(R⁵)R⁷NHR⁵, N(R⁵)R⁷Q, OR⁷OH, OR⁷OR⁵, OR⁷NH₂, OR⁷NHR⁵, OR⁷Q, OC(O)R⁵, NHC(O)R⁵, NHC(O)NHR⁵, OR⁶C(O)R⁵, NHR⁶C(O)R⁵, C(O)R⁵, C(O)OR⁵, C(O)NHR⁵, C(O)Q, R⁶C(O)H, R⁶C(O)R⁵, R⁶C(O)OH, R⁶C(O)OR⁵, R⁶C(O)NH₂, R⁶C(O)NHR⁵, R⁶C(O)Q, R⁶OC(O)R⁵, R⁶OC(O)NH₂, R⁶OC(O)NHR⁵, R⁶OC(O)Q and YR⁸, wherein Y is independently selected from C(O), C(O)O, OC(O), C(O)NH, NHC(O), NHSO₂, SO₂NH, C(OH)H, O(C(R⁹)₂)_{q}, S(O)ₘ(C(R⁹)₂)_{q}, NH(C(R⁹)₂)_{q}, NR¹⁰(C(R⁹)₂)_{q}, (C(R⁹)₂)_{q}, (C(R⁹)₂)_{q}O, (C(R⁹)₂)_{q}S(O)ₘ, (C(R⁹)₂)_{q}NH, (C(R⁹)₂)_{q}NR¹⁰, C≡C, cis and trans CH═CH and cycloalkyl of 3 to 10 carbon atoms, or A is a heteroaryl ring having 5 or 6 atoms containing 1 to 4 heteroatoms, which may be the same or different, selected from N, O and S wherein the heteroaryl ring may be optionally substituted with 1 to 4 substituents which may be the same or different selected from H, J, NO₂, NH₂, OH, SH, CN, COOH, CONH₂, NHC(O)NH₂, C(O)H, CF₃, OCF₃, R⁵, OR⁵, NHR⁵, Q, S(O)ₘR⁵, NHSO₂R⁵, R₆OH, R₆OR₅, R₆NH₂, R⁶NHR⁵, R⁶Q, R⁶SH, R⁶S(O)ₘR⁵, NHR⁷OH, NHR⁷OR⁵, N(R⁵)R⁷OH, N(R⁵)R⁷OR⁵, NHR⁷NH₂, NHR⁷NHR⁵, NHR⁷Q, N(R⁵)R⁷NH₂, N(R⁵)R⁷NHR⁵, N(R⁵)R⁷Q, OR⁷OH, R⁷OR⁵, OR⁷NH₂, OR⁷NHR⁵, OR⁷Q, OC(O)R⁵, NHC(O)R⁵, NHC(O)NHR⁵, R⁶C(O)R⁵, NHR⁶C(O)R⁵, C(O)R⁵, C(O)OR⁵, C(O)NHR⁵, C(O)Q, R⁶C(O)H, R⁶C(O)R⁵, R⁶C(O)OH, R⁶C(O)OR⁵, R⁶C(O)NH₂, R⁶C(O)NHR⁵, R⁶C(O)Q, R⁶OC(O)R⁵, R⁶OC(O)NH₂, R⁶OC(O)NHR⁵, R⁶OC(O)Q and YR⁸, wherein Y is independently selected from C(O), C(O)O, OC(O), C(O)NH, NHC(O), NHSO₂, SO₂NH, C(OH)H, O(C(R⁹)₂)_{q}, S(O)ₘ(C(R⁹)₂)_{q}, NH(C(R⁹)₂)_{q}, NR¹⁰(C(R⁹)₂)_{q}, (C(R⁹)₂)_{q}O, (C(R⁹)₂)_{q}S(O)ₘ, (C(R⁹)₂)_{q}NH, (C(R⁹)₂)_{q}NR¹⁰, C≡C, cis and trans CH═CH and cycloalkyl of 3 to 10 carbon atoms, or A is a bicyclic heteroaryl ring system having 8 to 20 atoms containing 1 to 4 heteroatoms which may be the same or different selected from N, O and S wherein the bicyclic heteroaryl ring system may be optionally substituted with 1 to 4 substituents which may be the same or different selected from H, J, NO₂, NH₂, OH, SH, CN, COOH, CONH₂, NHC(O)NH₂, C(O)H, CF₃, OCF₃, R⁵, OR⁵, NHR⁵, Q, S(O)ₘR⁵, NHSO₂R⁵, R₆OH, R₆OR₅, R₆NH₂, R⁶NHR⁵, R⁶Q, R⁶SH, R⁶S(O)ₘR⁵, NHR⁷OH, NHR⁷OR⁵, N(R⁵)R⁷OH, N(R⁵)R⁷OR⁵, NHR⁷NH₂, NHR⁷NHR⁵, NHR⁷Q, N(R⁵)R⁷NH₂, N(R⁵)R⁷NHR⁵, N(R⁵)R⁷Q, OR⁷OH, OR⁷OR⁵, OR⁷NH₂, OR⁷NHR⁵, OR⁷Q, OC(O)R⁵, NHC(O)R⁵, NHC(O)NHR⁵, OR⁶C(O)R⁵, NHR⁶C(O)R⁵, C(O)R⁵, C(O)OR⁵, C(O)NHR⁵, C(O)Q, R⁶C(O)H, R⁶C(O)R⁵, R⁶C(O)OH, R⁶C(O)OR⁵, R⁶C(O)NH₂, R⁶C(O)NHR⁵, R⁶C(O)Q, R⁶OC(O)R⁵, R⁶OC(O)NH₂, R⁶OC(O)NHR⁵, R⁶OC(O)Q and YR⁸, wherein Y is independently selected from C(O), C(O)O, OC(O), C(O)NH, NHC(O), NHSO₂, SO₂NH, C(OH)H, O(C(R⁹)₂)_{q}, S(O)ₘ(C(R⁹)₂)_{q}, NH(C(R⁹)₂)_{q}, NR¹⁰(C(R⁹)₂)_{q}, (C(R⁹)₂)_{q}, (C(R⁹)₂)_{q}O, (C(R⁹)₂)_{q}S(O)ₘ, (C(R⁹)₂)_{q}NH, (C(R⁹)₂)_{q}NR¹⁰, C≡C, cis and trans CH═CH and cycloalkyl of 3 to 10 carbon atoms, or A and —YR⁸ may be taken together to form a tricyclic ring system, J is selected from F and Cl, m is 0, 1 or 2, q is 0, 1, 2, 3, 4 or 5, s is 0, 1, 2 or 3, t is 1, 2, 3, 4, 5, 6, 7, 8, 9, 10, 11, or 12, R⁵ is a monovalent group wherein each R⁵ is independently selected from alkyl of 1-6 carbons, alkenyl of 2-6 carbon atoms, or alkynyl of 2-6 carbon atoms, or when two R⁵ are present on a nitrogen atom they may together form a hetrocyclic ring, R⁶ is a divalent group selected from alkyl of 1-6 carbons, alkenyl of 2-6 carbon atoms, or alkynyl of 2-6 carbon atoms, R⁷ is a divalent alkyl group of 2-6 carbon atoms, R⁸ is a cycloalkyl ring of 3-7 carbons that may be optionally substituted with one or more alkyl groups of 1 to 6 carbons, or R⁸ is a phenyl or heteroaryl ring, that can be fused to an additional phenyl or heteroaryl ring, wherein heteroaryl is as previously defined, and may be optionally substituted with 1 to 4 substituents selected from the group consisting of -Ph, —CH₂Ph, —NHPh, OPh, —S(O)ₘPh, J, —NO₂, —NH₂, —OH, —SH, —CN, —COOH, —CONH₂, —NHC(O)NH₂, —C(O)H, —CF₃, —OCF₃, —R⁵, —OR⁵, —NHR⁵, —NR⁵R⁵, —S(O)ₘR⁵, —NHSO₂R⁵, —R¹¹, —OR¹¹, —NHR¹¹, —R⁶OH, —R⁶OR⁵, —R⁶NH₂, —R⁶NHR⁵, —R⁶NR⁵R⁵, —R⁶SH, —R⁶S(O)ₘR⁵, —NHR⁷OH, —NHR⁷OR⁵, —N(R⁵)R⁷OH, —N(R⁵)R⁷OR⁵, —NHR⁷NH₂, —NHR⁷NHR⁵, —NHR⁷NR⁵R⁵, —N(R⁵)R⁷NH₂, —N(R⁵)R⁷NHR⁵, —N(R⁵)R⁷NHR⁵R⁵, —OR⁷OH, —OR⁷OR⁵, —OR⁷NH₂, —OR⁷NHR⁵, —OR⁷NR⁵R⁵, —OC(O)R⁵, —NHC(O)R⁵, —NHC(O)NHR⁵, —OR⁶C(O)R⁵, —NHR⁶C(O)R⁵, —C(O)R⁵, —C(O)OR⁵, —C(O)NHR⁵, C(O)NR⁵R⁵, —R⁶C(O)H, —R⁶C(O)R⁵, —R⁶C(O)OH, —R⁶C(O)OR⁵, —R⁶C(O)NH₂, —R⁶C(O)NHR⁵, —R⁶C(O)NR⁵R⁵, —R⁶OC(O)R⁵, —R⁶OC(O)NH₂, —R⁶OC(O)NHR⁵ and —R⁶OC(O)NR⁵R⁵, R⁹ is independently H, F or R⁵, R¹⁰ is an alkyl of 1-6 carbon atoms, R¹⁵ is independently selected from a group consisting of H, —R⁵, —R¹¹, —(CR⁹₂)_{q}Ph, —(CR⁹₂)_{q}—C₂-C₉ heteroaryl, —(CR⁹₂)_{q}—C₂-C₉ heterocycle, —(CR⁹₂)_{q}OH, —(CR⁹₂)_{q}OR¹⁰, (CR⁹₂)_{q}NH₂, —(CR⁹₂)_{q}NHR¹⁰, —(CR⁹₂)_{q}R¹⁰, —(CR⁹₂)_{q}S(O)ₘR¹⁰, —(CR⁹₂)_{q}CO₂R¹⁰, —(CR⁹₂)_{q}CONHR¹⁰, —(CR⁹₂)_{q}CONR¹⁰R¹⁰, —(CR⁹₂)_{q}COR¹⁰, —(CR⁹₂)_{q}CO₂H, and —(CR⁹₂)_{q}CONH₂, and Q is NR⁵R⁵ and further provided that when each R⁵ is independently selected from C₁-C₁₂ alkyl and C₂-C₆ alkenyl, each R⁵ may optionally be taken together with the nitrogen atom to which they are attached to form a heterocyclic ring of 3 to 8 atoms, optionally containing 1 or 2 additional heteroatoms which may be the same or different selected from N, O and S, comprising the step of reacting a reagent of formula (II):

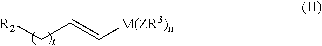

in the presence of Pd(0) metal with a compound of formula (III):

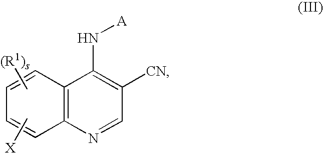

wherein X is selected from O-triflate, Br, I and Cl, M is Sn or B, Z is a bond, or an oxygen atom, with the proviso that Z can only be a bond when M is Sn and Z can only be an oxygen atom when M is B, u is 1, 2 or 3, and R³ is independently selected from H and alkyl of 1 to 12 carbon atoms, or two R³ groups taken together with Z and M can form a 3 to 8 membered ring, wherein the atoms of the ring can be selected from carbon, nitrogen, oxygen and sulfur, any of the substituents recited herein may be further substituted by groups selected from C₁-C₁₂ alkyl, F, Cl, C₁-C₁₂ fluoroalkyl, C₁-C₁₂ chloroalkyl, nitro, amino, hydroxyl, cyano, C₁-C₈ alkylamino, C₂-C₁₆ dialkylamino, C₁-C₁₂ alkoxy, C₁-C₁₂ fluoroalkoxy, C₁-C₁₂ chloroalkoxy, —S—C₁-C₁₂ alkyl, —SH, —S—C₁-C₁₂ fluoroalkyl, —S—C₁-C₁₂-alkyl, chloro C₆-C₁₂ aryl, C₆-C₁₂ aryloxy, —S—C₆-C₁₂ aryl, C₂-C₉ heteroaryl, C₂-C₉ heteroaryloxy, —S—C₂-C₉ heteroaryl and C₁-C₈ acyl, or salts thereof.

The present invention is also directed to a method of preparing compounds of formula (IV):

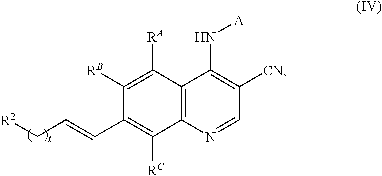

wherein A is selected from phenyl and C₂-C₉ heteroaryl, any of which may be substituted by substituents selected from H, F, Cl, alkoxy of 1 to 4 carbon atoms, alkyl of 1 to 4 carbon atoms, hydroxyl, fluoroalkyl of 1 to 4 carbon atoms, chloroalkyl of 1 to 4 carbon atoms, C₆-C₁₂ aryloxy, C₂-C₉ heteroaryloxy, —S-alkenyl of 1 to 4 carbon atoms, —S—C₆-C₁₂aryl, and —S—C₂-C₉ heteroaryl, R^{A}, R^{B} and R^{C} are independently selected from H, alkyl of 1 to 4 carbon atoms, alkoxy of 1 to 4 carbon atoms, F, Cl and CF₃, t is 1, 2, 3, 4, 5, 6, 7, 8, 9 or 10, and R² is selected from OH, C₁-C₄ alkyl —C(O)O—, alkylamino of 1 to 4 carbon atoms, dialkylamino of 2 to 8 carbons, C₆-C₁₂ aryl, cycloalkyl of 3 to 8 carbon atoms, C₂-C₉ heterocycloalkyl and (alkyl)₃Si—O— containing 3 to 12 carbon atoms, comprising the step of reacting a reagent of formula (II):

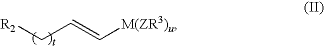

in the presence of a source of Pd(0) metal with a compound of formula (V):

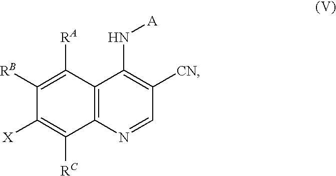

wherein, X is selected from O-triflate, Br, I and Cl, M is Sn or B, Z is a bond or an oxygen atom, with the proviso that Z is a bond when M is Sn and Z is oxygen when M is B, u is 1, 2 or 3, and R³ is independently selected from H and alkyl of 1 to 12 carbon atoms, or two R³ groups taken together with Z and M can form a 3 to 8 membered ring, wherein the atoms of the ring are selected from carbon, nitrogen, oxygen and sulfur, or salts thereof.

Another aspect of this invention is a method of preparing compounds of formula (VI):

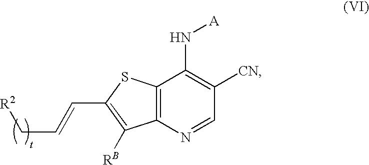

wherein A is selected from phenyl and C₂-C₉ heteroaryl, any of which may be substituted by substituents selected from H, F, Cl, alkoxy of 1 to 4 carbon atoms, alkyl of 1 to 4 carbon atoms, hydroxyl, fluoroalkyl of 1 to 4 carbon atoms, chloroalkyl of 1 to 4 carbon atoms, C₆-C₁₂ aryloxy, C₂-C₉ heteroaryloxy, —S-alkenyl of 1 to 4 carbon atoms, —S—C₆-C₁₂ aryl, and —S—C₂-C₉ heteroaryl, R^{B} is selected from H, F, Cl, alkyl of 1 to 4 carbon atoms, alkoxy of 1 to 4 carbon atoms, fluoroalkyl of 1 to 4 carbon atoms, chloroalkyl of 1 to 4 carbon atoms, OH, SH and —S-alkyl of 1 to 4 carbon atoms, t is 1 or 2, R² is selected from OH, C₁-C₄ alkyl-C(O)O—, alkylamino of 1 to 4 carbon atoms, dialkylamino of 2 to 8 carbons, C₆-C₁₂ aryl, cycloalkyl of 3 to 8 carbon atoms, C₁-C₉ heterocycloalkyl and (alkyl)₃Si—O— containing 3 to 12 carbon atoms, comprising the step of reacting a reagent of formula (II):

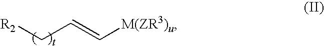

in the presence of a source of Pd(0) metal with a compound of formula (VII):

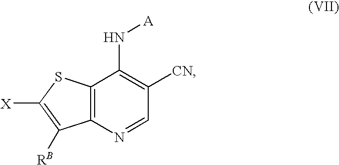

wherein X is selected from O-triflate, Br, I or Cl, M is Sn or B, Z is a bond or an oxygen atom, with the proviso that Z is a bond when M is Sn and Z is oxygen when M is B, u is 1, 2 or 3, and R³ is independently selected from H and alkyl of 1 to 12 carbon atoms, or two R³ groups taken together with Z and M can form a 3 to 8 membered heterocyclic ring, or salts thereof.

### DETAILED DESCRIPTION

The present invention is directed to methods of synthesizing compounds of formulas (I), (IV) and (VI) by reacting a compound of (III), (V) and (VII), respectively, with a vinyl boronic ester, or acid, or a vinyl stannane, of formula (II), in the presence of a catalytic amount of palladium metal.

One of the important features of this invention is that the coupling of a vinyl boronic ester or a vinyl stannane with a compound of formula (III), (V) or (VII) occurs stereoselectively, wherein the E-isomer is the predominate product.

For purposes of this invention the term “alkyl” includes either straight or branched alkyl moieties. The length of a straight alkyl moiety can be from 1 to 12 carbon atoms, but is preferably 1 to 8 carbon atoms, and more preferably 1 to 4 carbon atoms. Branched alkyl moieties can contain 3 to 12 carbon atoms. These alkyl moieties may be unsubstituted or substituted. The term “alkenyl” refers to a substituted or unsubstituted radical aliphatic hydrocarbon containing one double bond and includes alkenyl moieties of both straight, preferably of 2 to 6 carbon atoms and branched, preferably of 2 to 6 carbon atoms. Such alkenyl moieties may exist in the E or Z configurations; the compounds of this invention include both configurations. The term “alkynyl” includes substituted and unsubstituted alkynyl moieties of both straight chain containing 2 to 6 carbon atoms and branched containing 2 to 6 carbon atoms having at least one triple bond. The term “cycloalkyl” refers to substituted or unsubstituted alicyclic hydrocarbon groups having 3 to 12 carbon atoms and includes but is not limited to: cyclopropyl, cyclobutyl, cyclopentyl, cyclohexyl, cycloheptyl, norbornyl, or adamantyl. Most preferably the cycloalkyl group contains 3 to 6 carbon atoms.

For purposes of this invention the term “aryl” is defined as an aromatic hydrocarbon moiety and may be substituted or unsubstituted. An aryl may be selected from but not limited to, the group consisting of: phenyl, α-naphthyl, β-naphthyl, biphenyl, anthryl, tetrahydronaphthyl, phenanthryl, fluorenyl, indanyl, biphenylenyl, acenaphthenyl, acenaphthylenyl, or phenanthrenyl groups. Preferably an aryl group contains 6 to 12 carbon atoms.

For purposes of this invention the term “heteroaryl” is defined as an aromatic heterocyclic ring system (monocyclic or bicyclic) and may be substituted or unsubstituted where the heteroaryl moieties are five or six membered rings containing 1 to 4 heteroatoms selected from the group consisting of S, N, and O, and include but are not limited to: (1) furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, pyrrolidinyl; (2) a bicyclic aromatic heterocycle where a phenyl, pyridine, pyrimidine or pyridizine ring is: (i) fused to a 6-membered aromatic (unsaturated) heterocyclic ring having one nitrogen atom; (ii) fused to a 5 or 6-membered aromatic (unsaturated) heterocyclic ring having two nitrogen atoms; (iii) fused to a 5-membered aromatic (unsaturated) heterocyclic ring having one nitrogen atom together with either one oxygen or one sulfur atom; or (iv) fused to a 5-membered aromatic (unsaturated) heterocyclic ring having one heteroatom selected from O, N or S. Preferably a heteroaryl moiety contains 2 to 9 carbon atoms, and more preferably contains a total of 5 or 6 atoms.

For purposes of this invention the term “heterocycloalkyl” refers to a substituted or unsubstituted alicyclic ring system (monocyclic or bicyclic) wherein the heterocycloalkyl moieties are 3 to 12 membered rings containing 1 to 6 heteroatoms selected from the group consisting of S, N, and O. Examples include, but are not limited to, 1,3-dioxolane, pyrroline, pyrrolidine, imidazoline, imidazolidine, pyrazoline, pyrazolidine, piperidine, 1,4-dioxane, morpholine, thiomorpholine, and piperazine. Typically, such moieties contain 1 to 9 carbon atoms.

For the purposes of this invention the term “heterocycle” is defined as being either a heteroaryl or heterocycloalkyl, as defined herein.

For the purposes of this invention the term “alkoxy” is defined as alkyl-O—; the term “aryloxy” is defined as aryl-O—; the term “heteroaryloxy” is defined as heteroaryl-O—; wherein alkyl, aryl, and heteroaryl are as defined above.

The terms “alkylamino” and “dialkylamino” refer to moieties with one or two alkyl groups, respectively, wherein the alkyl chain is 1 to 8 carbons, more preferably 1 to 4 carbon atoms, and the groups may be the same or different. The terms alkylaminoalkyl and dialkylaminoalkyl refer, respectively, to alkylamino and dialkylamino moieties with one or two alkyl groups (the same or different) bonded to the nitrogen atom, which is attached to an alkyl group of 1 to 8 carbon atoms.

“Acyl” is a radical of the formula —C═O)-alkyl, —(C═O)-aryl, or —(C═O)-perfluoroalkyl wherein the alkyl radical or perfluoroalkyl radical is 1 to 8 carbon atoms and the aryl radical is as defined herein; preferred examples include but are not limited to, acetyl, propionyl, butyryl, trifluoroacetyl.

The terms “fluoroalkyl” and “chloroalkyl” refer to an alkyl radical that is further substituted by at least one fluorine or chlorine atom, respectively, and may be fully substituted, for example, —CF₃. The terms “fluoroalkoxy” and “chloroalkoxy” refer to an alkoxy radical that is further substituted by at least one fluorine or chlorine atom, respectively, and may be fully substituted, for example, —OCF₃.

The term “substituent” is used herein to refer to an atom radical, a functional group radical or a moiety radical that replaces a hydrogen radical on a molecule. Unless expressly stated otherwise, it should be assumed that any of the substituents may be optionally substituted with one or more groups selected from: alkyl, F, Cl, fluoroalkyl, chloroalkyl, nitro, amino, hydroxyl, cyano, alkylamino, dialkylamino, alkoxy, fluoroalkoxy, chloroalkoxy, —S-alkyl, —SH, —S-fluoroalkyl, —S-chloroalkyl, aryl, aryloxy, —S-aryl, heteroaryl, heteroaryloxy, —S-heteroaryl or acyl.

For the purposes of this invention the term “substituted” refers to where a hydrogen radical on a molecule has been replaced by another atom radical, a functional group radical or a moiety radical; these radicals being generally referred to as “substituents.”

Compounds made by the method of the present invention may contain an asymmetric carbon atom and some of the compounds of this invention may contain one or more asymmetric centers and may thus give rise to stereoisomers, such as enantiomers and diastereomers. While shown without respect to stereochemistry in Formulas (I), (IV) and (VI), the present invention includes the synthesis of all the individual possible stereoisomers; as well as the racemic mixtures and other mixtures of R and S stereoisomers (scalemic mixtures which are mixtures of unequal amounts of enantiomers) and salts thereof. It should be noted that stereoisomers of the invention having the same relative configuration at a chiral center may nevertheless have different R and S designations depending on the substitution at the indicated chiral center.

For compounds made by the method of the present invention containing two chiral centers, four possible stereoisomers are possible; these four stereoisomers are classified as two racemic pairs of diastereomers. These compounds may be present as racemic diastereomers which would be designated following the convention described in the 1997 Chemical Abstracts Index Guide, Appendix IV (Columbus, Ohio) whereas the first cited chiral atom is designated R* and the next cited chiral atom is designated R* if it possesses the same chirality as the first cited stereocenter or S* if it possesses opposite chirality to the first cited stereocenter. Alternatively, these compounds of the invention may be present as non-racemic mixtures of two diastereomers owing to the existence of a predefined stereocenter. In these instances, the predefined stereocenter is assigned based on the Cahn-Ingold-Prelog System and the undefined stereocenter is designated R* to denote a mixture of both R and S stereoisomers at this center.

Compounds made by the method of the present invention are alkenes and therefore can be designated using the (E)-(Z) system. One skilled in the art will be familiar with this system of nomenclature. Where alkene compounds are disclosed without stereospecifity it is intended that both of the diastereomers are encompassed by the disclosure.

Compounds made by the method of the present invention may be formed as salts from addition of organic and inorganic acids. For example salts can be formed from the addition of acids, including but not limited to, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids.

### General Synthetic Pathways:

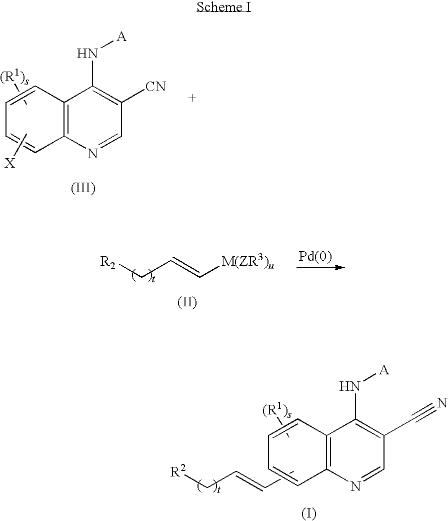

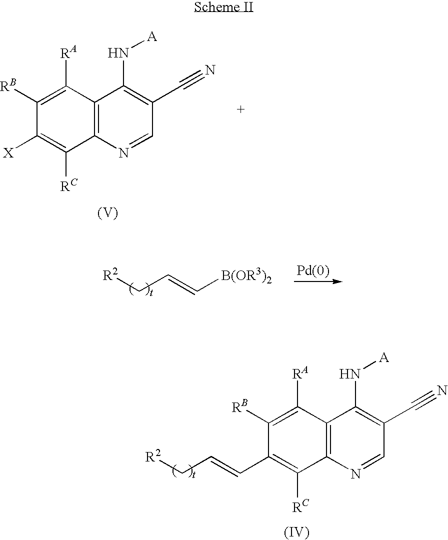

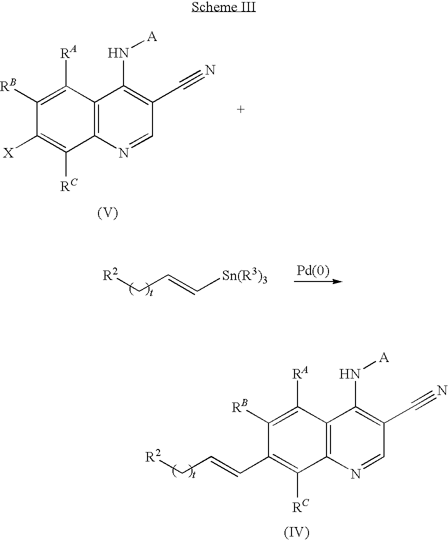

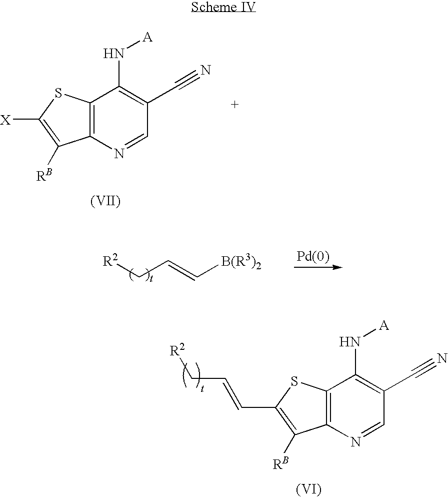

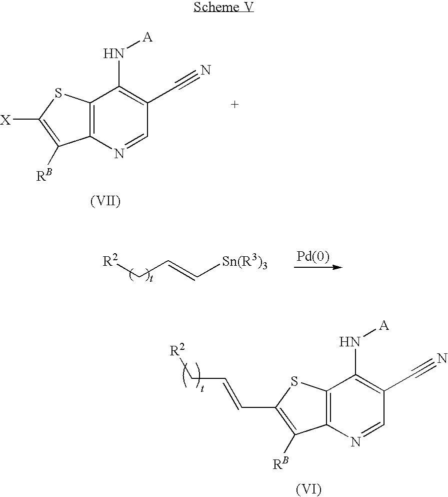

Scheme I illustrates the general synthetic pathway to compounds of formula (I) from starting 3-quinolinecarbonitriles of formula (III). The starting 3-quinolinecarbonitrile is coupled with a vinyl boronic ester or stannane of formula (II) in the presences of palladium metal in catalytic amounts, for example, Pd(PPh₃)₄. Where A, R¹-R³, X, s, t, u, m and Z are defined herein.

Palladium-mediated couplings of aryl halides with alk-1-enyl boranes are known by those skilled in the art. Such couplings were disclosed in Suzuki et al., J.C.S. Chem. Comm., 1979, No. 19, pp. 866-867, which is hereby incorporated by reference.

These coupling reactions are usually heated above room temperature, typically in the range of about 60° C. to about 120° C., but preferably about 80° C. to about 120° C. Preferably the temperature is raised to at least about 90° C., and more preferably to at least about 105° C. However the reaction can also be performed at temperatures as high as about 120° C.

Vinyl boronic esters or acids can be formed by hydroboration of the corresponding alkyne using 4,4,5,5-tetramethyl-[1,3,2]dioxaborolane and a catalytic amount of bis(cyclopentadienyl)zirconium chloride hydrate. This method of preparation was disclosed in Pereira and Siebnik, Organicmetallics 1995, 14, pp. 3127-3128, which is hereby incorporated by reference.

Vinyl stannanes can be prepared from the corresponding alkyne by reacting the alkyne with (alkyl)₃Sn, for example, tributylstannane, and a catalytic amount of AIBN. This method of preparing vinyl stannanes was disclosed in Jung et al., Tetrahedron Letters, Vol. 23 (38), pp. 3851-3854, 1982, which is hereby incorporated by reference.

This reaction can be carried out in a variety of solvents. One skilled in the art would be familiar with suitable solvents or mixtures of solvents appropriate for this reaction. Preferred solvents include N-methyl-2-pyrrolidone (NMP), toluene, benzene, toluene/ethanol/water (10:1:1), DMF, THF and DMF/THF (50:50).

In one embodiment of the present invention A is phenyl or substituted phenyl in the compounds of formulas (I) and (III).

In another embodiment of the present invention R¹ is selected from H, F, Cl and CH₃O in the compounds of formulas (I) and (III).

In yet another embodiment of the present invention R² is selected from morpholinyl, OH, CH₃C(O)O—, pyrrolidinyl, piperidinyl, n-methyl piperazinyl, n-ethylpiperazinyl, 4-(N-pyrrolidinyl)piperidinyl, 2-tetrahydropyranoxy, (CH₃)₃CSi(CH₃)₂O— and —NR¹⁹R²⁰. A more preferred embodiment is where R² is —NR¹⁹R²⁰.

Another embodiment of the present invention is where M is Sn and Z is a bond, or alternatively, where M is B and Z is O.

Scheme II shows the more specific synthetic method of synthesizing compounds of formula (IV) by reacting the starting 3-quinolinecarbonitrile of formula (V) with a vinyl boronic ester in the presence of a catalytic amount of palladium metal. The preferred solvent for this reaction is the mixture of toluene, ethanol and water (10:1:1). More specific reaction conditions are described under Method I in the General Methods section of this application.

Scheme III shows the more specific synthetic method to compounds of formula (IV) by reacting the starting 3-quinolinecarbonitrile of formula (V) with a vinyl stannane in the presence of a catalytic amount of palladium metal. The most preferred solvent for this reaction is NMP. More specific reaction conditions are described under Method II in the General Methods section herein.

In one embodiment of the present invention A is phenyl, which may be substituted, in compounds of formulas (IV) and (V). It is also preferable that A be substituted by H, Cl, OCH₃ or —S-heteroaryl.

In another embodiment of the present invention R^{A} and R^{C} are H in compounds of formulas (IV) and (V).

Another embodiment of the present invention is where R² is dialkylamino in compounds of formula (IV).

In yet another embodiment of the present invention M is Sn and Z is a bond, or alternatively, M is B and Z is oxygen.

Scheme IV shows the general method of the present invention for synthesizing 2-alkenyl-5-thienopyridinecarbonitriles of formula (VI) by coupling the starting 5-thienopyridinecarbonitrile of formula (VII) with a vinyl boronic ester, or acid, in the presence of a catalytic amount of palladium metal. The most preferred solvent for this reaction is the mixture of toluene, ethanol and water (10:1:1). This method is analogous to the one disclosed under Scheme II and therefore more specific conditions can be found under Method I of the General Methods section herein.

Scheme V shows the general method for synthesizing compounds of formula (VI) by reacting the starting 5-thienopyrioline carbonitrile of formula (VII) with a vinyl stannane in the presence of a catalytic amount of palladium metal. A preferred solvent for this reaction is NMP. The method is analogous to the method shown in Scheme III and therefore the same conditions described under Method II in the General Method section herein are applicable.

In one embodiment of the present invention A is phenyl, which may be substituted, in compounds of formulas (VI) and (VII). It is also preferable that A be substituted by H, Cl, OCH₃ or —S-heteroaryl.

In another embodiment of the present invention R^{B} is H in compounds of formulas (VI) and (VII).

Another embodiment of the present invention is where R² is dialkylamino in compounds of formula (VI).

In yet another embodiment of the present invention M is Sn and Z is a bond, or alternatively, M is B and Z is oxygen.

Similarly, the couplings illustrated in Schemes II-V are usually performed at a temperature above room temperature, typically in the range of about 60° C. to about 120° C., but preferably about 80° C. to about 120° C. Preferably the temperature is raised to at least about 90° C. and more preferably to at least about 105° C. However the reactions can also be performed at temperatures as high as about 120° C.

### General Methods:

### Method I

### 4-[(2,4-Dichloro-5-methoxyphenyl)amino]-6-methoxy-7-[(1E)-4-(4-methylpiperazin-1-yl)but-1-enyl]quinoline-3-carbonitrile

To a mixture of 1-but-3-ynyl-4-methyl-piperazine (1.85 g, 14.4 mmol) (the preparation of which was described in International Publication No. WO 2002/002558) and 4,4,5,5-tetramethyl-[1,3,2]dioxaborolane (1.46 g, 9.6 mmol) was added bis(cyclopentadienyl)zirconium chloride hydride (124 mg, 0.48 mmol). The resulting mixture was stirred at room temperature for 24 hours and was diluted with toluene/ethanol/water (80 mL/8 mL/8 mL). 3-Cyano-4-(2,4-dichloro-5-methoxyanilino)-6-methoxy-7-quinolinyl trifluoromethanesulfonate (2.50 g, 4.70 mmol) and Pd(PPh₃)₄ (285 mg, 0.238 mmol) were added. The reaction mixture was heated at 90° C. for 4 hours and partitioned between saturated aqueous NaHCO₃ and CH₂Cl₂. The combined organics were dried over Na₂SO₄, concentrated and purified by silica gel flash column chromatography (10:1 CH₂Cl₂-MeOH) to give 1.92 g of off-white solid, mp 142-143° C., MS (ESI) m/z 526.1.

### Method II

### 4-({3-Chloro-4-[(1-methyl-1H-imidazol-2-yl)thio]phenyl}amino)-7-[(1E)-5-(diethylamino)pent-1-enyl]quinoline-3-carbonitrile

A mixture of 7-bromo-4-{3-chloro-4-[(1-methyl-1H-imidazol-2-yl)sulfanyl]anilino}-3-quinolinecarbonitrile (377 mg, 0.80 mmol), (0.50 g, 1.12 mmol), diethyl[E-5-(tributylstannyl)-4-penten-1-yl]amine (0.48 g, 1.12 mmol) (the preparation of was which disclosed in International Publication No. WO 2004/033419) and NMP (4.0 mL) was treated under nitrogen with Pd(PPh₃)₄ (92 mg, 0.08 mmol) and stirred at 105° C. for 3 h. The cooled mixture was partitioned with CH₂Cl₂ and aqueous NaHCO₃. The organic layer was washed with H₂O, dried and concentrated. The residue was triturated with 1:1 hexane-Et₂O to remove NMP and then chromatographed on silica gel with 10:1 CH₂Cl₂-MeOH to give an off-white solid, mp 220-225° C. (dec). MS (ES+) m/z 533.1 (M+H)⁺¹.

### Method III

### (2E)-3-[4-({3-chloro-4-[(1-methyl-1H-imidazol-2-yl)thio]phenyl}amino)-3-cyanoquinolin-7-yl]prop-2-enyl acetate

A solution of 4-({3-chloro-4-[(1-methyl-1H-imidazol-2-yl)thio]phenyl}amino)-7-[(1E)-3-hydroxyprop-1-enyl]quinoline-3-carbonitrile (1.87 g, 3.2. mmol) (Example 4), 24 ml of Ac₂O, and 24 ml of HOAc was stirred at 50° C. for 19 h, concentrated in the presence of toluene, and stirred in aqueous NaHCO₃. The resulting solid was dissolved in 60:30:1-EtOAc—HOAc and filtered through a pad of silica gel. The residue obtained on evaporation was stirred in McOH—H₂O containing NaHCO₃, filtered, washed with H₂O, and dried to give a light yellow solid, mp 181-193° C. (dec); m/z 492.1⁻ (M+H)⁺¹.

### Method IV

### 4-({3-chloro-4-[(1-methyl-1H-imidazol-2-yl)thio]phenyl}amino)-7-[(1E)-3-(diethylamino)prop-1-enyl]quinoline-3-carbonitrile

A mixture of (2E)-3-[4-({3-chloro-4-[(1-methyl-1H-imidazol-2-yl)thio]phenyl}amino)-3-cyanoquinolin-7-yl]prop-2-enyl acetate (196 mg, 0.40 mmol), diethylamine (165 μl, 1.6 mmol), and 0.80 ml of NMP under nitrogen was treated with Pd(PPh₃)₄ (46 mg, 0.04 mmol) and stirred at 25° C. for 1 h. The mixture was stirred with aqueous NaHCO₃ and 4:1 hexane-EtOAc and filtered. The solid product was dissolved in 10:1 CH₂Cl₂-MeOH and passed through a short column of silica gel. The washings which contained product were evaporated to give 93 mg of off-white solid, mp 223-228° C.; MS (ES+) m/z 505.0 (M+H)⁺¹.

### Method V

### 4-[(2,4-dichloro-5-methoxyphenyl)amino]-7-[(1E)-4-(4-ethylpiperazin-1-yl)but-1-enyl]-6-methoxyquinoline-3-carbonitrile

To a mixture of 4-[(2,4-dichloro-5-methoxyphenyl)amino]-7-[(1E)-4-hydroxybut-1-enyl]-6-methoxyquinoline-3-carbonitrile (150 mg, 0-351 mmol) and Et₃N (178 mg, 1.76 mmol) in DMF/THF (2 mL/2 mL) was added methanesulfonyl chloride (121 mg, 1.05 mmol) in THF (1 mL). The resulting mixture was stirred at room temperature overnight and was then treated with N-ethylpiperazine (200 mg, 1.76 mmol) at 75° C. for 48 h. The cooled reaction mixture was partitioned between water and CH₂Cl₂. The combined organics were dried, concentrated and purified by silica gel flash column chromatograph to give 95 mg of off-white solid, mp 129-131° C.; MS (ESI) m/z 540.1.

### Method VI

### N-[E-4-(tributylstannyl)-3-buten-1-yl]pyrrolidine

To a stirred solution of E-4-(tributylstannyl)-3-buten-1-yl tosylate (1.55 g, 3.0 mmol) (the preparation of which was disclosed in Heterocycles (1997), 46, 523 and is hereby incorporated by reference) in 3.0 ml of THF at 25° C. was added pyrrolidine (1.0 ml, 12 mmol). After 18 h the volatile materials were evaporated under vacuum, and the residue was partitioned with aqueous NaHCO₃ and 1:1 hexane-Et₂O. The organic layer was washed with H₂O, dried and evaporated to give an oil; ¹H NMR (CDCl₃) δ 5.95 (m, 2H, vinyl), 2.53 (m, 8H), 2.37 (m, 4H), 1.78 (m, 6H), 1.49 (m, 6H), 1.30 (m, 6H), 0.89 (t, J=7.3 Hz, 9H).

### EXAMPLES

### Example 1

### 4-[(2,4-dichlorophenyl)amino]-7-[(1E)-5-morpholin-4-ylpent-1-enyl]quinoline-3-carbonitrile

The title compound is prepared using a procedure analogous to Method II from 7-bromo-4-(2,4-dichloro-phenylamino)-quinoline-3-carbonitrile and 4-[(E)-5-(tributylstannyl)-4-pentenyl]morpholine, mp 142-144° C.; MS (ESI) m/z 467.1.

### Example 2

### 4-[(2,4-dichlorophenyl)amino]-7-[(1E)-6-morpholin-4-ylhex-1-enyl]quinoline-3-carbonitrile

The title compound is prepared using a procedure analogous to Method II from 7-bromo-4-(2,4-dichloro-phenylamino)-quinoline-3-carbonitrile and 4-[(5E)-6-(tributylstannyl)hex-5-enyl]morpholine, mp 139-140° C.; MS (ESI) m/z 481.2.

### Example 3

### 4-[(2,4-dichloro-5-methoxyphenyl)amino]-6-methoxy-7-[(1E)-5-morpholin-4-ylpent-1-enyl]quinoline-3-carbonitrile

The title compound is prepared using a procedure analogous to Method II from 3-cyano-4-(2,4-dichloro-5-methoxyanilino)-6-methoxy-7-quinolinyl trifluoromethanesulfonate and 4-[(E)-5-(tributylstannyl)-4-pentenyl]morpholine, mp 110-112° C.; MS (ESI) m/z 527.2.

### Example 4

### 4-({3-chloro-4-[(1-methyl-1H-imidazol-2-yl)thio]phenyl}amino)-7-[(1E)-3-hydroxyprop-1-enyl]quinoline-3-carbonitrile

The title compound is prepared using a procedure analogous to Method II from 7-bromo-4-{3-chloro-4-[(1-methyl-1H-imidazol-2-yl)sulfanyl]anilino}-3-quinolinecarbonitrile and 3-(E)-tributylstannanyl-prop-2-en-1-ol, mp 220-240° C.; MS (ESI) m/z 448.

### Example 5

### 4-({3-chloro-4-[(1-methyl-1H-imidazol-2-yl)thio]phenyl}amino)-7-[(1E)-4-hydroxybut-1-enyl]quinoline-3-carbonitrile

The title compound is prepared using a procedure analogous to Method II from 7-bromo-4-{3-chloro-4-[(1-methyl-1H-imidazol-2-yl)sulfanyl]anilino}-3-quinolinecarbonitrile and 4-(E)-tributylstannanyl-but-3-en-1-ol, mp 205-210° C.; MS (ESI) m/z 462.2.

### Example 6

### (2E)-3-[4-({3-chloro-4-[(1-methyl-1H-imidazol-2-yl)thio]phenyl}amino)-3-cyanoquinolin-7-yl]prop-2-enyl acetate

The title compound is prepared as described in Method III from 4-({3-chloro-4-[(1-methyl-1H-imidazol-2-yl)thio]phenyl}amino)-7-[(1E)-3-hydroxyprop-1-enyl]quinoline-3-carbonitrile, 181-193° C.; MS (ESI) m/z 490.1.

### Example 7

### 4-({3-chloro-4-[(1-methyl-1H-imidazol-2-yl)thio]phenyl}amino)-7-[(1E)-3-morpholin-4-ylprop-1-enyl]quinoline-3-carbonitrile

The title compound is prepared using a procedure analogous to Method IV from (2E)-3-[4-({3-chloro-4-[(1-methyl-1H-imidazol-2-yl)thio]phenyl}amino)-3-cyanoquinolin-7-yl]prop-2-enyl acetate and morphoine, mp 235-240° C.; MS (ESI) m/z 517.1.

### Example 8

### 4-({3-chloro-4-[(1-methyl-1H-imidazol-2-yl)thio]phenyl}amino)-7-[(1E)-4-(diethylamino)but-1-enyl]quinoline-3-carbonitrile

The title compound is prepared using a procedure analogous to Method II from 7-bromo-4-{3-chloro-4-[(1-methyl-1H-imidazol-2-yl)sulfanyl]anilino}-3-quinolinecarbonitrile and N,N-diethyl-N-[(3E)-4-(tributylstannyl)but-3-enyl]amine, mp 200-210° C.; MS (ESI) m/z 517.1.

### Example 9

### 4-({3-chloro-4-[(1-methyl-1H-imidazol-2-yl)thio]phenyl}amino)-7-[(1E)-4-(dimethylamino)but-1-enyl]quinoline-3-carbonitrile

The title compound is prepared using a procedure analogous to Method VI from toluene-4-sulfonic acid 4-{4-[3-chloro-4-(1-methyl-1H-imidazol-2-ylsulfanyl)-phenylamino]-3-cyano-quinolin-7-yl}-but-3-enyl ester and dimethylamine, mp 191-198° C.; MS (ESI) m/z 489. Toluene-4-sulfonic acid 4-{4-[3-chloro-4-(1-methyl-1H-imidazol-2-ylsulfanyl)-phenylamino]-3-cyano-quinolin-7-yl}-but-3-enyl ester was prepared using a procedure analogous to Method II from 7-bromo-4-{3-chloro-4-[(1-methyl-1H-imidazol-2-yl)sulfanyl]anilino}-3-quinolinecarbonitrile and E-4-(Tributylstannyl)-3-buten-1-yl tosylate.

### E-4-(Tributylstannyl)-3-buten-1-yl tosylate

To a stirred solution E-(4-hydroxybuten-1-yl)tributylstannane (5.42 g, 15 mmol, the preparation of which was disclosed in J. Org. Chem. 1998, 63, pp. 7811) in 30 ml of 2,6-lutidine was added tosyl chloride (8.58 g, 45 mmol) at 25° C. After 20 h the mixture was treated with 30 ml of H₂O and 5 ml of pyridine with cooling. After 15 minutes at 25° C. the mixture was partitioned with DCM and aqueous NaHCO₃. The organic layer was washed with H₂O, dried and concentrated to give an oil; ¹H NMR (DMSO-d₆) δ 7.76 (d, J=8.0 Hz, 2H), 7.48 (d, J=8.0 Hz, 2H).

### Example 10

### 4-({3-chloro-4-[(1-methyl-1H-imidazol-2-yl)thio]phenyl}amino)-7-[(1E)-4-morpholin-4-ylbut-1-enyl]quinoline-3-carbonitrile

The title compound was prepared using a procedure analogous to Method II from 7-bromo-4-{3-chloro-4-[(1-methyl-1H-imidazol-2-yl)sulfanyl]anilino}-3-quinolinecarbonitrile and 4-[(E)-5-(tributylstannyl)-4-pentenyl]morpholine, mp 232-238° C.; MS (ESI) m/z 531.

### Example 11

### 4-({3-chloro-4-[(1-methyl-1H-imidazol-2-yl)thio]phenyl}amino)-7-[(1E)-3-(diethylamino)prop-1-enyl]quinoline-3-carbonitrile

The title compound is prepared using a procedure analogous to Method IV from (2E)-3-[4-({3-chloro-4-[(1-methyl-1H-imidazol-2-yl)thio]phenyl}amino)-3-cyanoquinolin-7-yl]prop-2-enyl acetate and diethylamine, mp 223-228° C.; MS (ESI) m/z 503.

### Example 12

### 4-({3-chloro-4-[(1-methyl-1H-imidazol-2-yl)thio]phenyl}amino)-7-[(1E)-4-pyrrolidin-1-ylbut-1-enyl]quinoline-3-carbonitrile

The title compound is prepared using a procedure analogous to Method VI from toluene-4-sulfonic acid 4-{4-[3-chloro-4-(1-methyl-1H-imidazol-2-ylsulfanyl)-phenylamino]-3-cyano-quinolin-7-yl}-but-3-enyl ester and pyrrolidine, mp 185-191° C.; MS (ESI) m/z 515.1.

### Example 13

### 4-({3-chloro-4-[(1-methyl-1H-imidazol-2-yl)thio]phenyl}amino)-7-[(1E)-3-dimethylamino)prop-1-enyl]quinoline-3-carbonitrile

The title compound is prepared using a procedure analogous to Method IV from (2E)-3-[4-({3-chloro-4-[(1-methyl-1H-imidazol-2-yl)thio]phenyl}amino)-3-cyanoquinolin-7-yl]prop-2-enyl acetate and dimethylamine, mp 157-165° C.; MS (ESI) m/z 475.

### Example 14

### 4-({3-chloro-4-[(1-methyl-1H-imidazol-2-yl)thio]phenyl}amino)-7-[(1E)-4-piperidin-1-ylbut-1-enyl]quinoline-3-carbonitrile

The title compound is prepared using a procedure analogous to Method VI from toluene-4-sulfonic acid 4-{4-[3-chloro-4-(1-methyl-1H-imidazol-2-ylsulfanyl)-phenylamino]-3-cyano-quinolin-7-yl}-but-3-enyl ester and piperidine, mp 205-210° C.; MS (ESI) m/z 529.

### Example 15

### 4-({3-chloro-4-[(1-methyl-1H-imidazol-2-yl)thio]phenyl}amino)-7-[(1E)-3-pyrrolidin-1-ylprop-1-enyl]quinoline-3-carbonitrile

The title compound is prepared using a procedure analogous to Method IV from (2E)-3-[4-({3-chloro-4-[(1-methyl-1H-imidazol-2-yl)thio]phenyl}amino)-3-cyanoquinolin-7-yl]prop-2-enyl acetate and pyrrolidine, mp 182-190° C.; MS (ESI) m/z 501.1.

### Example 16

### 4-({3-chloro-4-[(1-methyl-1H-imidazol-2-yl)thio]phenyl}amino)-7-[(1E)-4-hydroxybut-1-enyl]-6-methoxyquinoline-3-carbonitrile

The title compound is prepared using a procedure analogous to Method II from 4-({3-chloro-4-[(1-methyl-1H-imidazol-2-yl)thio]phenyl}amino)-3-cyano-6-methoxyquinolin-7-yl trifluoromethanesulfonate and 4-(E)-tributylstannanyl-but-3-en-1-ol, mp 273-278° C.; MS (ESI) m/z 492.

### Example 17

### 4-({3-chloro-4-[(1-methyl-1H-imidazol-2-yl)thio]phenyl}amino)-7-[(1E)-5-pyrrolidin-1-ylpent-1-enyl]quinoline-3-carbonitrile

The title compound is prepared using a procedure analogous to Method VI from toluene-4-sulfonic acid 4-{4-[3-chloro-4-(1-methyl-1H-imidazol-2-ylsulfanyl)-phenylamino]-3-cyano-quinolin-7-yl}-pent-3-enyl ester and pyrrolidine, mp 217-222° C.; MS (ESI) m/z 529.2. Toluene-4-sulfonic acid 4-{4-[3-chloro-4-(1-methyl-1H-imidazol-2-ylsulfanyl)-phenylamino]-3-cyano-quinolin-7-yl}-pent-3-enyl ester was prepared using a procedure analogous to Method II from 7-bromo-4-{3-chloro-4-[(1-methyl-1H-imidazol-2-yl)sulfanyl]anilino}-3-quinolinecarbonitrile and E-4-(tributylstannyl)-3-pent-1-yl tosylate.

### Example 18

### 4-({3-chloro-4-[(1-methyl-1H-imidazol-2-yl)thio]phenyl}amino)-7-[(1E)-4-(diethylamino)but-1-enyl]-6-methoxyquinoline-3-carbonitrile

The title compound is prepared using a procedure analogous to Method VI from toluene-4-sulfonic acid 4-{4-[3-chloro-4-(1-methyl-1H-imidazol-2-ylsulfanyl)-phenylamino]-6-methoxy-3-cyano-quinolin-7-yl}-but-3-enyl ester and diethylamine, mp 194-202° C.; MS (ESI) m/z 547.2.

### Example 19

### 4-({3-chloro-4-[(1-methyl-1H-imidazol-2-yl)thio]phenyl}amino)-6-methoxy-7-[(1E)-4-pyrrolidin-1-yl but-1-enyl]quinoline-3-carbonitrile

The title compound is prepared using a procedure analogous to Method II 4-({3-chloro-4-[(1-methyl-1H-imidazol-2-yl)thio]phenyl}amino)-3-cyano-6-methoxyquinolin-7-yl trifluoromethanesulfonate and 1-methyl-4-(4-tributylstannanyl-but-3-enyl)-piperazine mp 230-234° C.; MS (ESI) m/z 545.1.

### Example 20

### 4-({3-chloro-4-[(1-methyl-1H-imidazol-2-yl)thio]phenyl}amino)-7-[(1E)-4-(4-methylpiperazin-1-yl)but-1-enyl]quinoline-3-carbonitrile

The title compound was prepared using a procedure analogous to Method II from 7-bromo-4-{3-chloro-4-[(1-methyl-1H-imidazol-2-yl)sulfanyl]anilino}-3-quinolinecarbonitrile and 1-methyl-4-(E)-(4-tributylstannanyl-but-3-enyl)-piperazine, mp 225-235° C.; MS (ESI) m/z 544.2.

### Example 21

### 4-({3-chloro-4-[(1-methyl-1H-imidazol-2-yl)thio]phenyl}amino)-6-methoxy-7-[(1E)-4-(4-methylpiperazin-1-yl)but-1-enyl]quinoline-3-carbonitrile

The title compound is prepared using a procedure analogous to Method II from 4-({3-chloro-4-[(1-methyl-1H-imidazol-2-yl)thio]phenyl}amino)-3-cyano-6-methoxyquinolin-7-yl trifluoromethanesulfonate and 1-methyl-4-(E)-(4-tributylstannanyl-but-3-enyl)-piperazine, mp 223-203° C.; MS (ESI) m/z 574.2.

### Example 22

### 4-({3-chloro-4-[(1-methyl-1H-imidazol-2-yl)thio]phenyl}amino)-7-[(1E)-4-(dimethylamino)but-1-enyl]-6-methoxyquinoline-3-carbonitrile

The title compound is prepared using a procedure analogous to Method II from 4-({3-chloro-4-[(1-methyl-1H-imidazol-2-yl)thio]phenyl}amino)-3-cyano-6-methoxyquinolin-7-yl trifluoromethanesulfonate and dimethyl-4-(E)-(tributylstannanyl-but-3-enyl)-amine, mp 215-225° C.

### Example 23

### 4-[(2,4-dichloro-5-methoxyphenyl)amino]-7-[(1E)-4-morpholin-4-ylbut-1-enyl]quinoline-3-carbonitrile

The title compound is prepared using a procedure analogous to Method II from 7-bromo-4-(2,4-dichloro-5-methoxyanilino)-3-quinolinecarbonitrile and 4-[(E)-5-(tributylstannyl)-4-butenyl]morpholine, mp 88-89° C.; MS (ESI) m/z 483.1.

### Example 24

### 4-[(2,4-dichloro-5-methoxyphenyl)amino]-6-methoxy-7-[(1E)-4-morpholin-4-ylbut-1 enyl]quinoline-3-carbonitrile

The title compound is prepared using a procedure analogous to Method II from 3-cyano-4-(2,4-dichloro-5-methoxyanilino)-6-methoxy-7-quinolinyl trifluoromethanesulfonate and 4-[(E)-5-(tributylstannyl)-4-butenyl]morpholine, mp 141-143° C.; MS (ESI) m/z 513.1.

### Example 25

### 4-({3-chloro-4-[(1-methyl-1H-imidazol-2-yl)thio]phenyl}amino)-7-[(1E)-5-(diethylamino)pent-1-enyl]quinoline-3-carbonitrile

The title compound was prepared using a procedure analogous to Method II from 7-bromo-4-{3-chloro-4-[(1-methyl-1H-imidazol-2-yl)sulfanyl]anilino}-3-quinolinecarbonitrile and diethyl-(E)-(5-tributylstannanyl-pent-4-enyl)-amine, mp 220-225° C.; MS (ESL) m/z 531.1.

### Example 26

### 4-({3-chloro-4-[(1-methyl-1H-imidazol-2-yl)thio]phenyl}amino)-7-[(1E)-5-(diethylamino)pent-1-enyl]-6-methoxyquinoline-3-carbonitrile

The title compound is prepared using a procedure analogous to Method II from 4-({3-chloro-4-[(1-methyl-1H-imidazol-2-yl)thio]phenyl}amino)-3-cyano-6-methoxyquinolin-7-yl trifluoromethanesulfonate and diethyl-(E)-(5-tributylstannanyl-pent-4-enyl)-amine, mp 229-233° C.; MS (ESI) m/z 561.1.

### Example 27

### 4-({3-chloro-4-[(1-methyl-1H-imidazol-2-yl)thio]phenyl}amino)-7-[(1E)-5-(4-methylpiperazin-1-yl)pent-1-enyl]quinoline-3-carbonitrile

The title compound was prepared using a procedure analogous to Method II from 7-bromo-4-{3-chloro-4-[(1-methyl-1H-imidazol-2-yl)sulfanyl]anilino}-3-quinolinecarbonitrile and 1-methyl-4-(E)-(5-tributylstannanyl-pent-4-enyl)-piperazine, mp 219-227° C.; MS (ESI) m/z 558.1.

### Example 28

### 4-({3-chloro-4-[(1-methyl-1H-imidazol-2-yl)thio]phenyl}amino)-6-methoxy-7-[(1E)-5-(4-methylpiperazin-1-yl)pent-1-enyl]quinoline-3-carbonitrile

The title compound is prepared using a procedure analogous to Method II from 4-({3-chloro-4-[(1-methyl-1H-imidazol-2-yl)thio]phenyl}amino)-3-cyano-6-methoxyquinolin-7-yl trifluoromethanesulfonate and 1-methyl-4-(E)-(5-tributylstannanyl-pent-4-enyl)-piperazine, mp 215-221° C.; MS (ESI) m/z 588.1.

### Example 29

### 4-[(2,4-dichloro-5-methoxyphenyl)amino]-6-methoxy-7-[(1E)-4-(4-methylpiperazin-1-yl)but-1-enyl]quinoline-3-carbonitrile

The title compound was prepared as described in Method I from 3-cyano-4-(2,4-dichloro-5-methoxyanilino)-6-methoxy-7-quinolinyl trifluoromethanesulfonate, 1-but-3-ynyl-4-methyl-piperazine and 4,4,5,5-tetramethyl-[1,3,2]dioxaborolane or using a procedure analogous to Method II from 3-cyano-4-(2,4-dichloro-5-methoxyanilino)-6-methoxy-7-quinolinyl trifluoromethanesulfonate and 1-methyl-4-(E)-(4-tributylstannanyl-but-3-enyl)-piperazine, mp 142-143° C.; MS (ESI) m/z 526.1.

### Example 30

### 4-[(2,4-dichloro-5-methoxyphenyl)amino]-7-[(1E)-4-hydroxybut-1-enyl]-6-methoxyquinoline-3-carbonitrile

The title compound was prepared using a procedure analogous to Method I from 3-cyano-4-(2,4-dichloro-5-methoxyanilino)-6-methoxy-7-quinolinyl trifluoromethanesulfonate, tert-butyl-but-3-ynyloxy-dimethyl-silane and 4,4,5,5-tetramethyl-[1,3,2]dioxaborolane followed by acidic hydrolysis, mp 186-188° C.; MS (ESI) m/z 444.1.

### Example 31

### 7-[(1E)-4-morpholin-4-ylbut-1-enyl]-4-[(3,4,5-trimethoxyphenyl)amino]quinoline-3-carbonitrile

The title compound was prepared using a procedure analogous to Method II from 7-bromo-4-(3,4,5-trimethoxyanilino)-3-quinolinecarbonitrile and 4-[(E)-5-(tributylstannyl)-4-pentenyl]morpholine, mp 128-130° C.; MS (ESI) m/z 475.2.

### Example 32

### 4-[(2,4-dichloro-5-methoxyphenyl)amino]-6-methoxy-7-[(1E)-3-morpholin-4-ylprop-1-enyl]quinoline-3-carbonitrile

The title compound was prepared as described in Method II from 3-cyano-4-(2,4-dichloro-5-methoxyanilino)-6-methoxy-7-quinolinyl trifluoromethanesulfonate and 4-[(E)-5-(tributylstannyl)-4-propenyl]morpholine, mp 105-106° C.; MS (ESI) m/z 499.1.

### Example 33

### 6-methoxy-7-[(1E)-4-(4-methylpiperazin-1-yl)but-1-en-1-yl]-4-[(3,4,5-trimethoxyphenyl)amino]quinoline-3-carbonitrile

The title compound was prepared as described in Method I from 3-cyano-6-methoxy-4-[(3,4,5-trimethoxyphenyl)amino]quinolin-7-yl trifluoromethanesulfonate, 1-but-3-ynyl-4-methyl-piperazine and 4,4,5,5-tetramethyl-[1,3,2]dioxaborolane, mp 122-124° C.; MS (ESI) m/z 518.2.

### Example 34

### 4-[(2,4-dichloro-5-methoxyphenyl)amino]-6-methoxy-7-[(1E)-4-piperidin-1-ylbut-1-enyl]quinoline-3-carbonitrile

The title compound is prepared using a procedure analogous to Method V from 4-[(2,4-dichloro-5-methoxyphenyl)amino]-7-[(1E)-4-hydroxybut-1-enyl]-6-methoxyquinoline-3-carbonitrile and piperidine, mp 140-142° C.; MS (ESI) m/z 511.1.

### Example 35

### 4-[(2,4-dichlorophenyl)amino]-7-[(1E)-4-morpholin-4-ylbut-1-enyl]quinoline-3-carbonitrile

The title compound is prepared using a procedure analogous to Method II from 7-bromo-4-(2,4-dichloro-phenylamino)-quinoline-3-carbonitrile and 4-[(5E)-6-(tributylstannyl)but-5-enyl]morpholine, mp 129-131° C.; MS (ESI) m/z 453.1

### Example 36

### 4-[(2,4-dichlorophenyl)amino]-7-[(1E)-3-morpholin-4-ylprop-1-enyl]quinoline-3-carbonitrile

The title compound is prepared using a procedure analogous to Method II from 7-bromo-4-(2,4-dichloro-phenylamino)-quinoline-3-carbonitrile and 4-[(5E)-6-(tributylstannyl)prop-5-enyl]morpholine, mp 175-176° C.; MS (ESI) m/z 439.1.

### Example 37

### 4-({3-chloro-4-[(1-methyl-1H-imidazol-2-yl)thio]phenyl}amino)-7-[(1E)-3-(4-methylpiperazin-1-yl)prop-1-enyl]quinoline-3-carbonitrile

The title compound is prepared using a procedure analogous to Method IV from (2E)-3-[4-({3-chloro-4-[(1-methyl-1H-imidazol-2-yl)thio]phenyl}amino)-3-cyanoquinolin-7-yl]prop-2-enyl acetate and N-methylpiperazine, MS (ESI) m/z 530.

### Example 38

### 4-[(2,4-dichloro-5-methoxyphenyl)amino]-6-methoxy-7-[(1E)-6-morpholin-4-ylhex-1-enyl]quinoline-3-carbonitrile

The title compound is prepared using a procedure analogous to Method II from 7-bromo-4-(2,4-dichloro-phenylamino)-quinoline-3-carbonitrile and 4-[(5E)-6-(tributylstannyl)hex-5-enyl]morpholine, mp 99-100° C.; MS (ESI) m/z 541.1.

### Example 39

### 4-[(2,4-dichlorophenyl)amino]-7-[(1E)-11-morpholin-4-ylundec-1-enyl]quinoline-3-carbonitrile

The title compound is prepared using a procedure analogous to Method II from 7-bromo-4-(2,4-dichloro-phenylamino)-quinoline-3-carbonitrile and 4-[(5E)-6-(tributylstannyl)undec-5-enyl]morpholine, mp 105-106° C.; MS (ESI) m/z 551.2.

### Example 40

### 4-[(2,4-dichloro-5-methoxyphenyl)amino]-6-methoxy-7-[(1E)-11-morpholin-4-ylundec-1-enyl]quinoline-3-carbonitrile

The title compound is prepared using a procedure analogous to Method II from 3-cyano-4-(2,4-dichloro-5-methoxyanilino)-6-methoxy-7-quinolinyl trifluoromethanesulfonate and 4-[(5E)-6-(tributylstannyl)undec-5-enyl]morpholine, mp 98-99° C.; MS (ESI) m/z 611.3;

### Example 41

### 4-({3-chloro-4-[(1-methyl-1H-imidazol-2-yl)thio]phenyl}amino)-7-[(1E)-3-piperidin-1-ylprop-1-enyl]quinoline-3-carbonitrile

The title compound is prepared using a procedure analogous to Method IV from (2E)-3-[4-({3-chloro-4-[(1-methyl-1H-imidazol-2-yl)thio]phenyl}amino)-3-cyanoquinolin-7-yl]prop-2-enyl acetate and piperidine, MS (ESI) m/z 515.1.

### Example 42

### 4-({3-chloro-4-[(1-methyl-1H-imidazol-2-yl)thio]phenyl}amino)-7-[(1E)-3-(4-pyrrolidin-1-ylpiperidin-1-yl)prop-1-enyl]quinoline-3-carbonitrile

The title compound is prepared using a procedure analogous to Method IV from (2E)-3-[4-({3-chloro-4-[(1-methyl-1H-imidazol-2-yl)thio]phenyl}amino)-3-cyanoquinolin-7-yl]prop-2-enyl acetate and 4-pyrrolidin-1-yl-piperidine, MS (ESI) m/z 584.1.

### Example 43

### 4-({3-chloro-4-[(1-methyl-1H-imidazol-2-yl)thio]phenyl}amino)-7-[(1E)-3-(4-ethyl piperazin-1-yl)prop-1-enyl]quinoline-3-carbonitrile

The title compound is prepared using a procedure analogous to Method IV from (2E)-3-[4-({3-chloro-4-[(1-methyl-1H-imidazol-2-yl)thio]phenyl}amino)-3-cyanoquinolin-7-yl]prop-2-enyl acetate and N-ethylpiperazine, mp 232-236° C.; MS (ESI) m/z 544.1.

### Example 44

### 4-({3-chloro-4-[(1-methyl-1H-imidazol-2-yl)thio]phenyl}amino)-7-[(1E)-4-(4-ethylpiperazin-1-yl)but-1-enyl]quinoline-3-carbonitrile

The title compound was prepared using a procedure analogous to Method I from 7-bromo-4-{3-chloro-4-[(1-methyl-1H-imidazol-2-yl)sulfanyl]anilino}-3-quinolinecarbonitrile, 1-but-3-ynyl-4-methyl-piperazine and 4,4,5,5-tetramethyl-[1,3,2]dioxaborolane, mp 194-204° C.; MS (ESI) m/z 558.1.

### Example 45

### 4-({3-chloro-4-[(1-methyl-1H-imidazol-2-yl)thio]phenyl}amino)-7-[(1E)-4-(tetrahydro-2H-pyran-2-yloxy)but-1-enyl]quinoline-3-carbonitrile

The title compound was prepared using a procedure analogous to Method I from 7-bromo-4-{3-chloro-4-[(1-methyl-1H-imidazol-2-yl)sulfanyl]anilino}-3-quinolinecarbonitrile and 2-{[(3E)-4-(4,4,5,5-tetramethyl-1,3,2-dioxaborolan-2-yl)but-3-enyl]oxy}tetrahydro-2H-pyran, mp 217-220° C.; MS (ESI) m/z 546.1.

### Example 46

### 7-((1E)-4-{[tert-butyl(dimethyl)silyl]oxy}but-1-enyl)-4-[(2,4-dichloro-5-methoxyphenyl)amino]-6-methoxyquinoline-3-carbonitrile

The title compound was prepared using a procedure analogous to Method I from 3-cyano-4-(2,4-dichloro-5-methoxyanilino)-6-methoxy-7-quinolinyl trifluoromethanesulfonate, (3-butynyloxy)-(1,1-dimethylethyl)dimethylsilane and 4,4,5,5-tetramethyl-[1,3,2]dioxaborolane, mp 97-99° C.; MS (ESI+) m/z 558.1.

### Example 47

### 4-({3-chloro-4-[(1-methyl-1H-imidazol-2-yl)thio]phenyl}amino)-7-[(1E)-4-(4-pyrrolidin-1-ylpiperidin-1-yl)but-1-enyl]quinoline-3-carbonitrile

The title compound was prepared using a procedure analogous to Method I from 7-bromo-4-{3-chloro-4-[(1-methyl-1H-imidazol-2-yl)sulfanyl]anilino}-3-quinolinecarbonitrile, 1-but-3-ynyl-4-pyrrolidin-1-yl-piperidine and 4,4,5,5-tetramethyl-[1,3,2]dioxaborolane, mp 214-216° C.; MS (ESI+) m/z 598.2.

### Example 48

### 4-[(2,4-dichloro-5-methoxyphenyl)amino]-7-[(1E)-4-(4-methylpiperazin-1-yl)but-1-enyl]quinoline-3-carbonitrile

The title compound is prepared using a procedure analogous to Method II from 7-bromo-4-(2,4-dichloro-5-methoxyphenylamino)-quinoline-3-carbonitrile and 1-methyl-4-[(3E)-4-(tributylstannyl)but-3-enyl]piperazine, mp 152-154° C.; MS (ESI+) m/z 496.1.

### Example 49

### 4-({3-chloro-4-[(1-methyl-1H-imidazol-2-yl)thio]phenyl}amino)-6-fluoro-7-[(1E)-4-(4-methylpiperazin-1-yl)but-1-enyl]quinoline-3-carbonitrile

The title compound is prepared using a procedure analogous to Method II from 4-({3-chloro-4-[(1-methyl-1H-imidazol-2-yl)thio]phenyl}amino)-3-cyano-6-fluoroquinolin-7-yl trifluoromethanesulfonate and 1-methyl-4-(E)-(4-tributylstannanyl-but-3-enyl)-piperazine, mp 188-194° C.; MS (ESI+) m/z 562.1.

### Example 50

### 4-[(2,4-dichloro-5-methoxyphenyl)amino]-7-[(1E)-4-(dimethylamino)but-1-enyl]-6-methoxyquinoline-3-carbonitrile

The title compound is prepared using a procedure analogous to Method V from 4-[(2,4-dichloro-5-methoxyphenyl)amino]-7-[(1E)-4-hydroxybut-1-enyl]-6-methoxyquinoline-3-carbonitrile and dimethylamine, mp 119-121° C.; MS (ESI) m/z 471.1.

### Example 51

### 4-[(2,4-dichloro-5-methoxyphenyl)amino]-7-[(1E)-4-(4-ethylpiperazin-1-yl)but-1-enyl]-6-methoxyquinoline-3-carbonitrile

The title compound is prepared using a procedure analogous to Method V from 4-[(2,4-dichloro-5-methoxyphenyl)amino]-7-[(1E)-4-hydroxybut-1-enyl]-6-methoxyquinoline-3-carbonitrile and N-ethylpiperazine, mp 129-131° C.; MS (ESI) m/z 540.1.

### Example 52

### 7-[(1E)-4-hydroxybut-1-en-1-yl]-6-methoxy-4-[(3,4,5-trimethoxyphenyl)amino]quinoline-3-carbonitrile

The title compound was prepared using a procedure analogous to Method I from 4-[(3,4-5-trimethoxyphenyl)amino]-7-[(1E)-4-hydroxybut-1-enyl]-6-methoxyquinoline-3-carbonitrile, tert-butyl-but-3-ynyloxy-dimethyl-silane and 4,4,5,5-tetramethyl-[1,3,2]dioxaborolane followed by acidic hydrolysis, mp 184-185° C.; MS (ESI) m/z 436.1.

### Example 53

### 4-[(2,4-dichloro-5-methoxyphenyl)amino]-6-methoxy-7-[(1E)-4-pyrrolidin-1-ylbut-1-en-1-yl]quinoline-3-carbonitrile

The title compound is prepared using a procedure analogous to Method V from 4-[(2,4-dichloro-5-methoxyphenyl)amino]-7-[(1E)-4-hydroxybut-1-enyl]-6-methoxyquinoline-3-carbonitrile and pyrrolidine, mp 158-159° C.; MS (ESI) m/z 497.1.

### Example 54

### 7-({3-chloro-4-[(1-methyl-1H-imidazol-2-yl)thio]phenyl}amino)-2-[(1E)-4-pyrrolidin-1-ylbut-1-enyl]thieno[3,2-b]pyridine-6-carbonitrile

The title compound was prepared using a procedure analogous to Method I from 7-({3-chloro-4-[(1-methyl-1H-imidazol-2-yl)thio]phenyl}amino)-2-iodothieno[3,2-b]pyridine-6-carbonitrile, 1-but-3-ynyl-pyrrolidine and 4,4,5,5-tetramethyl-[1,3,2]dioxaborolane; MS (ESI+) m/z 521.1.

### Example 55

### 7-({3-chloro-4-[(1-methyl-1H-imidazol-2-yl)thio]phenyl}amino)-2-[(1E)-4-(4-pyrrolidin-1-ylpiperidin-1-yl)but-1-enyl]thieno[3,2-b]pyridine-6-carbonitrile

The title compound was prepared using a procedure analogous to Method I from 7-({3-chloro-4-[(1-methyl-1H-imidazol-2-yl)thio]phenyl}amino)-2-iodothieno[3,2-b]pyridine-6-carbonitrile, 1-but-3-ynyl-4-pyrrolidin-1-yl-piperidine and 4,4,5,5-tetramethyl-[1,3,2]dioxaborolane, mp 213-216° C.; MS (ESI+) m/z 604.2.

### Example 56

### 7-({3-chloro-4-[(1-methyl-1H-imidazol-2-yl)thio]phenyl}amino)-2-[(1E)-4-(4-ethylpiperazin-1-yl)but-1-enyl]thieno[3,2-b]pyridine-6-carbonitrile

The title compound was prepared using a procedure analogous to Method I from 7-({3-chloro-4-((1-methyl-1H-imidazol-2-yl)thio]phenyl}amino)-2-iodothieno[3,2-b]pyridine-6-carbonitrile, 1-but-3-ynyl-4-ethyl-piperazine and 4,4,5,5-tetramethyl-[1,3,2]dioxaborolane, mp 179-182° C. (dec.); MS (ESI+) m/z 564.1.

### Example 57

### 7-({3-chloro-4-[(1-methyl-1H-imidazol-2-yl)thio]phenyl}amino)-2-[(E)-4-(4-methylpiperazin-1-yl)but-1-en-1-yl]thieno[3,2-b]pyridine-6-carbonitrile

The title compound was prepared using a procedure analogous to Method I from 7-({3-chloro-4-[(1-methyl-1H-imidazol-2-yl)thio]phenyl}amino)-2-iodothieno[3,2-b]pyridine-6-carbonitrile, 1-but-3-ynyl-4-methyl-piperazine and 4,4,5,5-tetramethyl-[1,3,2]dioxaborolane, mp 160-163° C. (dec.); MS (ESI+) m/z 550.1.

### Example 58

### 7-({3-chloro-4-[(1-methyl-1H-imidazol-2-yl)thio]phenyl}amino)-2-[(1E)-3-(dimethylamino)prop-1-en-1-yl]thieno[3,2-b]pyridine-6-carbonitrile

The title compound was prepared using a procedure analogous to Method I from 7-({3-chloro-4-[(1-methyl-1H-imidazol-2-yl)thio]phenyl}amino)-2-iodothieno[3,2-b]pyridine-6-carbonitrile, but-3-ynyl-dimethyl-amine and 4,4,5,5-tetramethyl-[1,3,2]dioxaborolane, mp 208-210° C. (dec.); MS (ESI) m/z 481.1.

### Example 59

### 4-[(2,4-dichloro-5-methoxyphenyl)amino]-8-[(1E)-4-morpholin-4-ylbut-1-enyl]quinoline-3-carbonitrile

The title compound was prepared using a procedure analogous to Method II from 8-bromo-4-(2,4-dichloro-5-methoxyphenylamino)-quinoline-3-carbonitrile and 4-[(E)-5-(tributylstannyl)-4-butenyl]morpholine, mp 176-179° C.; MS (ESI) m/z 483.1.

### Example 60

### 4-[(2,4-dichlorophenyl)amino]-6-[(1E)-4-morpholin-4-ylbut-1-enyl]quinoline-3-carbonitrile

The title compound was prepared using a procedure analogous to Method II from 6-bromo-4-(2,4-dichlorophenylamino)-quinoline-3-carbonitrile and 4-[(E)-5-(tributylstannyl)-4-butenyl]morpholine, mp 115-116° C.; MS (ESI) m/z 453.1.

### Example 61

### 4-[(2,4-dichloro-5-methoxyphenyl)amino]-6-[(1E)-4-morpholin-4-ylbut-1-enyl]quinoline-3-carbonitrile

The title compound was prepared using a procedure analogous to Method II from 6-bromo-4-(2,4-dichloro-5-methoxyphenylamino)-quinoline-3-carbonitrile and 4-[(E)-5-(tributylstannyl)-4-butenyl]morpholine, mp 95-96° C.; MS (ESI) m/z 483.1.

### Example 62

### 4-[(2,4-dichlorophenyl)amino]-6-[(1E)-4-(4-methylpiperazin-1-yl)but-1-enyl]quinoline-3-carbonitrile

The title compound was prepared using a procedure analogous to Method II from 6-bromo-4-(2,4-dichlorophenylamino)-quinoline-3-carbonitrile and 1-methyl-4-(E)-(4-tributylstannanyl-but-3-enyl)-piperazine, mp 97-98° C.; MS (ESI) m/z 466.1.

### Example 63

### 6-[(1E)-4-morpholin-4-ylbut-1-enyl]-4-[(3,4,5-trimethoxyphenyl)amino]quinoline-3-carbonitrile

The title compound was prepared using a procedure analogous to Method II from 6-bromo-4-(3,4,5-trimethoxyphenylamino)-quinoline-3-carbonitrile and 4-[(E)-5-(tributylstannyl)-4-butenyl]morpholine, mp 86-87° C.; MS (ESI) m/z 475.2

1. A method for preparing compounds of formula (I): 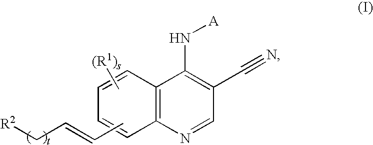
wherein:
R¹ is independently selected from H, alkyl of 1 to 6 carbon atoms, C₁-C₁₂ alkoxy, F, Cl and CF₃;
R² is selected from the group H, alkyl of 1 to 6 carbon atoms, OH, Cl, F, acetyl, —OSO₂—C₆-C₁₂ aryl, —OSO₂—C₁-C₁₂ alkyl, C₁-C₄ alkyl —C(O)O—, dialkylamino of 2 to 8 carbons, C₆-C₁₂ aryl, cycloalkyl of 3 to 8 carbon atoms, C₁-C₉ heterocycloalkyl, (alkyl)₃Si—O— containing 3 to 12 carbon atoms, any of which may be substituted and —NR¹⁹R²⁰, where R¹⁹ and R²⁰ can independently be H and alkyl of 1 to 6 carbon atoms, or R¹⁹ and R²⁰ taken together form a 3 to 8 membered substituted or unsubstituted heterocycle containing 1-3 heteroatoms selected from O, S, or N, and wherein said alkyl of 1 to 6 carbon atoms can be substituted with groups selected from C₁-C₆ alkylamino, C₂-C₁₂ dialkylamino, and a 3-8 membered heterocycle containing 1-3 heteroatoms selected from O, S, or N;
A is aryl of 6 to 12 carbon atoms optionally substituted with 1 to 4 substituents which are independently selected from H, J, NO₂, NH₂, OH, SH, CN, COOH, CONH₂, NHC(O)NH₂, C(O)H, CF₃, OCF₃, R⁵, OR⁵, NHR⁵, Q, S(O)ₘR⁵, NHSO₂R⁵, R₆OH, R₆OR₅, R₆NH₂, R⁶NHR⁵, R⁶Q, R⁶SH, R⁶S(O)ₘR⁵, NHR⁷OH, NHR⁷OR⁵, N(R⁵)R⁷OH, N(R⁵)R⁷OR⁵, NHR⁷NH₂, NHR⁷NHR⁵, NHR⁷Q, N(R⁵)R⁷NH₂, N(R⁵)R⁷NHR⁵, N(R⁵)R⁷Q, OR⁷OH, OR⁷OR⁵, OR⁷NH₂, OR⁷NHR⁵, OR⁷Q, OC(O)R⁵, NHC(O)R⁵, NHC(O)NHR⁵, OR⁶C(O)R⁵, NHR⁶C(O)R⁵, C(O)R⁵, C(O)OR⁵, C(O)NHR⁵, C(O)Q, R⁶C(O)H, R⁶C(O)R⁵, R⁶C(O)OH, R⁶C(O)OR⁵, R⁶C(O)NH₂, R⁶C(O)NHR⁵, R⁶C(O)Q, R⁶OC(O)R⁵, R⁶OC(O)NH₂, R⁶OC(O)NHR⁵, R⁶OC(O)Q and YR⁸, wherein Y is independently selected from C(O), C(O)O, OC(O), C(O)NH, NHC(O), NHSO₂, SO₂NH, C(OH)H, O(C(R⁹)₂)_{q}, S(O)ₘ(C(R⁹)₂)_{q}, NH(C(R⁹)₂)_{q}, NR¹⁰(C(R⁹)₂)_{q}, (C(R⁹)₂)_{q}, (C(R⁹)₂)_{q}O, (C(R⁹)₂)_{q}S(O)ₘ, (C(R⁹)₂)_{q}NH, (C(R⁹)₂)_{q}NR¹⁰, C≡C, cis and trans CH═CH and cycloalkyl of 3 to 10 carbon atoms; or
A is a heteroaryl ring having 5 or 6 atoms containing 1 to 4 heteroatoms or particularly 1 or 2 heteroatoms which may be the same or different, selected from N, O and S wherein the heteroaryl ring may be optionally substituted with 1 to 4 substituents which may be the same or different selected from H, J, NO₂, NH₂, OH, SH, CN, COOH, CONH₂, NHC(O)NH₂, C(O)H, CF₃, OCF₃, R⁵, OR⁵, NHR⁵, Q, S(O)ₘR⁵, NHSO₂R⁵, R₆OH, R₆OR₅, R₆NH₂, R⁶NHR⁵, R⁶Q, R⁶SH, R⁶S(O)ₘR⁵, NHR⁷OH, NHR⁷OR⁵, N(R⁵)R⁷OH, N(R⁵)R⁷OR⁵, NHR⁷NH₂, NHR⁷NHR⁵, NHR⁷Q, N(R⁵)R⁷NH₂, N(R⁵)R⁷NHR⁵, N(R⁵)R⁷Q, OR⁷OH, R⁷OR⁵, OR⁷NH₂, OR⁷NHR⁵, OR⁷Q, OC(O)R⁵, NHC(O)R⁵, NHC(O)NHR⁵, R⁶C(O)R⁵, NHR⁶C(O)R⁵, C(O)R⁵, C(O)OR⁵, C(O)NHR⁵, C(O)Q, R⁶C(O)H, R⁶C(O)R⁵, R⁶C(O)OH, R⁶C(O)OR⁵, R⁶C(O)NH₂, R⁶C(O)NHR⁵, R⁶C(O)Q, R⁶OC(O)R⁵, R⁶OC(O)NH₂, R⁶OC(O)NHR⁵, R⁶OC(O)Q and YR⁸, wherein Y is independently selected from C(O), C(O)O, OC(O), C(O)NH, NHC(O), NHSO₂, SO₂NH, C(OH)H, O(C(R⁹)₂)_{q}, S(O)ₘ(C(R⁹)₂)_{q}, NH(C(R⁹)₂)_{q}, NR¹⁰(C(R⁹)₂)_{q}, (C(R⁹)₂)_{q}O, (C(R⁹)₂)_{q}S(O)ₘ, (C(R⁹)₂)_{q}NH, (C(R⁹)₂)_{q}NR¹⁰, C≡C, cis and trans CH═CH and cycloalkyl of 3 to 10 carbon atoms; or
A is a bicyclic heteroaryl ring system having 8 to 20 atoms containing 1 to 4 heteroatoms which may be the same or different selected from N, O and S wherein the bicyclic heteroaryl ring system may be optionally substituted with 1 to 4 substituents which may be the same or different selected from H, J, NO₂, NH₂, OH, SH, CN, COOH, CONH₂, NHC(O)NH₂, C(O)H, CF₃, OCF₃, R⁵, OR⁵, NHR⁵, Q, S(O)ₘR⁵, NHSO₂R⁵, R₆OH, R₆OR₅, R₆NH₂, R⁶NHR⁵, R⁶Q, R⁶SH, R⁶S(O)ₘR⁵, NHR⁷OH, NHR⁷OR⁵, N(R⁵)R⁷OH, N(R⁵)R⁷OR⁵, NHR⁷NH₂, NHR⁷NHR⁵, NHR⁷Q, N(R⁵)R⁷NH₂, N(R⁵)R⁷NHR⁵, N(R⁵)R⁷Q, OR⁷OH, OR⁷OR⁵, OR⁷NH₂, OR⁷NHR⁵, OR⁷Q, OC(O)R⁵, NHC(O)R⁵, NHC(O)NHR⁵, OR⁶C(O)R⁵, NHR⁶C(O)R⁵, C(O)R⁵, C(O)OR⁵, C(O)NHR⁵, C(O)Q, R⁶C(O)H, R⁶C(O)R⁵, R⁶C(O)OH, R⁶C(O)OR⁵, R⁶C(O)NH₂, R⁶C(O)NHR⁵, R⁶C(O)Q, R⁶OC(O)R⁵, R⁶OC(O)NH₂, R⁶OC(O)NHR⁵, R⁶OC(O)Q and YR⁸, wherein Y is independently selected from C(O), C(O)O, OC(O), C(O)NH, NHC(O), NHSO₂, SO₂NH, C(OH)H, O(C(R⁹)₂)_{q}, S(O)ₘ(C(R⁹)₂)_{q}, NH(C(R⁹)₂)_{q}, NR¹⁰(C(R⁹)₂)_{q}, (C(R⁹)₂)_{q}, (C(R⁹)₂)_{q}O, (C(R⁹)₂)_{q}S(O)ₘ, (C(R⁹)₂)_{q}NH, (C(R⁹)₂)_{q}NR¹⁰, C≡C, cis and trans CH═CH and cycloalkyl of 3 to 10 carbon atoms; or
A and —YR⁸ may be taken together to form a tricyclic ring system;
J is selected from F and Cl;
m is 0, 1 or 2;
q is 0, 1, 2, 3, 4 or 5;
s is 0, 1, 2 or 3;
t is 1, 2, 3, 4, 5, 6, 7, 8, 9, 10, 11, or 12;
R⁵ is a monovalent group wherein each R⁵ is independently selected from alkyl of 1-6 carbons, alkenyl of 2-6 carbon atoms, or alkynyl of 2-6 carbon atoms, or when two R⁵ are present on a nitrogen atom they may together form a hetrocyclic ring;
R⁶ is a divalent group selected from alkyl of 1-6 carbons, alkenyl of 2-6 carbon atoms, or alkynyl of 2-6 carbon atoms;
R⁷ is a divalent alkyl group of 2-6 carbon atoms;
R⁸ is a cycloalkyl ring of 3-7 carbons that may be optionally substituted with one or more alkyl groups of 1 to 6 carbons; or
R⁸ is a phenyl or heteroaryl ring, that can be fused to an additional phenyl or heteroaryl ring, wherein heteroaryl is as previously defined, and may be optionally substituted with 1 to 4 substituents selected from the group consisting of -Ph, —CH₂Ph, —NHPh, OPh, —S(O)ₘPh, J, —NO₂, —NH₂, —OH, —SH, —CN, —COOH, —CONH₂, —NHC(O)NH₂, —C(O)H, —CF₃, —OCF₃, —R⁵, —OR⁵, —NHR⁵, —NR⁵R⁵, —S(O)ₘR⁵, —NHSO₂R⁵, —R¹¹, —OR¹¹, —NHR¹¹, —R⁶OH, —R⁶OR⁵, —R⁶NH₂, —R⁶NHR⁵, —R⁶NR⁵R⁵, —R⁶SH, —R⁶S(O)ₘR⁵, —NHR⁷OH, —NHR⁷OR⁵, —N(R⁵)R⁷OH, —N(R⁵)R⁷OR⁵, —NHR⁷NH₂, —NHR⁷NHR⁵, —NHR⁷NR⁵R⁵, —N(R⁵)R⁷NH₂, —N(R⁵)R⁷NHR⁵, —N(R⁵)R⁷NHR⁵R⁵, —OR⁷OH, —OR⁷OR⁵, —OR⁷NH₂, —OR⁷NHR⁵, —OR⁷NR⁵R⁵, —OC(O)R⁵, —NHC(O)R⁵, —NHC(O)NHR⁵, —OR⁶C(O)R⁵, —NHR⁶C(O)R⁵, —C(O)R⁵, —C(O)OR⁵, —C(O)NHR⁵, C(O)NR⁵R⁵, —R⁶C(O)H, —R⁶C(O)R⁵, —R⁶C(O)OH, —R⁶C(O)OR⁵, —R⁶C(O)NH₂, —R⁶C(O)NHR⁵, —R⁶C(O)NR⁵R⁵, —R⁶OC(O)R⁵, —R⁶OC(O)NH₂, —R⁶OC(O)NHR⁵ and —R⁶OC(O)NR⁵R⁵;
R⁹ is independently H, F or R⁵;
R¹⁰ is an alkyl of 1-6 carbon atoms;
R¹⁵ is independently selected from a group consisting of H, —R⁵, —R¹¹, —(CR⁹₂)_{q}Ph, —(CR⁹₂)_{q}—C₂-C₉ heteroaryl, —(CR⁹₂)_{q}C₂-C₉ heterocycle, —(CR⁹₂)_{q}OH, —(CR⁹₂)_{q}OR¹⁰, (CR⁹₂)_{q}NH₂, —(CR⁹₂)_{q}NHR¹⁰, —(CR⁹₂)_{q}R¹⁰, —(CR⁹₂)_{q}S(O)ₘR¹⁰, —(CR⁹₂)_{q}CO₂R¹⁰, —(CR⁹₂)_{q}CONHR¹⁰, —(CR⁹₂)_{q}CONR¹⁰R¹⁰, —(CR⁹₂)_{q}COR¹⁰, —(CR⁹₂)_{q}CO₂H, and —(CR⁹₂)_{q}CONH₂
Q is NR⁵R⁵ and further provided that when each R⁵ is independently selected from C₁-C₁₂ alkyl and C₂-C₆ alkenyl, each R⁵ may optionally be taken together with the nitrogen atom to which they are attached to form a heterocyclic ring of 3 to 8 atoms, optionally containing 1 or 2 additional heteroatoms which may be the same or different selected from N, O and S;
comprising the step of reacting a reagent of formula (II): 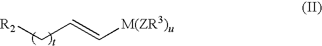
in the presence of Pd(0) metal with a compound of formula (III): 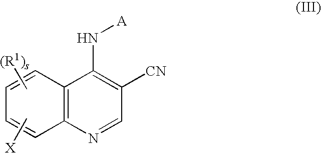
wherein:
X is selected from O-triflate, Br, I and Cl;
M is Sn or B;
Z is a bond, or an oxygen atom, with the proviso that Z can only be a bond when M is Sn and Z can only be an oxygen atom when M is B;
u is 1, 2 or 3; and
R³ is independently selected from H, alkyl of 1 to 12 carbon atoms, or two R³ groups taken together with Z and M can form a 3 to 8 membered ring, wherein the atoms of the ring can be selected from carbon, nitrogen, oxygen and sulfur;
any of the substituents recited herein may be further substituted with groups selected from C₁-C₁₂ alkyl, F, Cl, C₁-C₁₂ fluoroalkyl, C₁-C₁₂ chloroalkyl, nitro, amino, hydroxyl, cyano, C₁-C₈ alkylamino, C₂-C₁₆ dialkylamino, C₁-C₁₂ alkoxy, C₁-C₁₂ fluoroalkoxy, C₁-C₁₂ chloroalkoxy, —S—C₁-C₁₂ alkyl, —SH, —S—C₁-C₁₂ fluoroalkyl, —S—C₁-C₁₂ chloroalkyl, C₆-C₁₂ aryl, C₆-C₁₂ aryloxy, —S—C₆-C₁₆ aryl, C₂-C₉ heteroaryl, C₂-C₉ heteroaryloxy, —S—C₂-C₉ heteroaryl and C₁-C₈ acyl;
or salts thereof.

2. The method of claim 1, wherein A is phenyl or substituted phenyl.

3. The method of claim 1, wherein R¹ is selected from H, F, Cl and —OCH₃.

4. The method of claim 1, wherein R² is selected from morpholinyl, OH, CH₃C(O)O—, pyrrolidinyl, piperidinyl, n-methyl piperazinyl, n-ethylpiperazinyl, 4-(N-pyrrolidinyl)piperidinyl, 2-tetrahydropyranoxy and (CH₃)₃CSi(CH₃)₂O—.

5. The method of claim 4, wherein R² is —NR¹⁹R²⁰.

6. The method of claim 1, wherein M is Sn and Z is a bond.

7. The method of claim 1, wherein M is B and Z is O.

8. The method of claim 1, wherein the source of the Pd(0) metal is Pd(PPh₃)₄.

9. A method of preparing compounds of formula (IV): 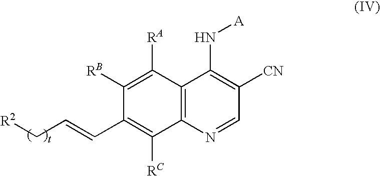
wherein:
A is selected from phenyl and C₂-C₉ heteroaryl, either of which may be substituted by one or more substituents selected from H, F, Cl, alkoxy of 1 to 4 carbon atoms, alkyl of 1 to 4 carbon atoms, hydroxyl, fluoroalkyl of 1 to 4 carbon atoms, chloroalkyl of 1 to 4 carbon atoms, C₆-C₁₂ aryloxy, C₂-C₉ heteroaryloxy, —S-alkenyl of 1 to 4 carbon atoms, —S—C₆-C₁₂ aryl, and —S—C₂-C₉ heteroaryl;
R^{A}, R^{B} and R^{C} are independently selected from H, alkyl of 1 to 4 carbon atoms, alkoxy of 1 to 4 carbon atoms, F, Cl and CF₃;
t is 1, 2, 3, 4, 5, 6, 7, 8, 9 or 10;
R² is selected from OH, C₁-C₄ alkyl —C(O)O—, alkylamino of 1 to 4 carbon atoms, dialkylamino of 2 to 8 carbons, C₆-C₁₂ aryl, cycloalkyl of 3 to 8 carbon atoms, C₁-C₉ heterocycloalkyl and (alkyl)₃Si—O— containing 3 to 12 carbon atoms, any of which may be substituted;
comprising the step of reacting a reagent of formula (II): 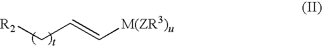
(II)
in the presence of a source of Pd(0) metal with a compound of formula (V): 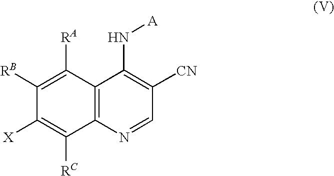
wherein:
X is selected from O-triflate, Br, I and Cl;
M is Sn or B;
Z is a bond or an oxygen atom, with the proviso that Z is a bond when M is Sn and Z is oxygen when M is B;
u is 1, 2 or 3; and
R³ is independently selected from H and alkyl of 1 to 12 carbon atoms, or two R³ groups taken together with Z and M can form a 3 to 8 membered ring, wherein the atoms of the ring are selected from carbon, nitrogen, oxygen and sulfur;
or salts thereof.

10. The method of claim 9, wherein A is phenyl, which may be substituted.

11. The method of claim 10, wherein R^{A} and R^{C} are H.

12. The method of claim 11, wherein A is substituted by H, Cl, OCH₃ or —S-heteroaryl.

13. The method of claim 9, wherein R² is dialkylamino.

14. The method of claim 9, wherein M is Sn and Z is a bond.

15. The method of claim 9, wherein M is B and Z is oxygen.

16. The method of claim 9, wherein the source of Pd(0) is Pd(PPh₃)₄.

17. A method of preparing compounds of formula (VI): 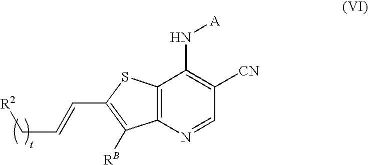
wherein:
A is selected from phenyl and C₂-C₉ heteroaryl, either of which may be substituted by one or more substituents selected from H, F, Cl, alkoxy of 1 to 4 carbon atoms, alkyl of 1 to 4 carbon atoms, hydroxyl, fluoroalkyl of 1 to 4 carbon atoms, chloroalkyl of 1 to 4 carbon atoms, C₆-C₁₂ aryloxy, C₂-C₉ heteroaryloxy, —S-alkenyl of 1 to 4 carbon atoms, —S—C₆-C₁₂ aryl, and —S—C₂-C₉ heteroaryl;
R^{B} is selected from H, F, Cl, alkyl of 1 to 4 carbon atoms, alkoxy of 1 to 4 carbon atoms, fluoroalkyl of 1 to 4 carbon atoms, chloroalkyl of 1 to 4 carbon atoms, OH, SH and —S-alkyl of 1 to 4 carbon atoms;
t is 1 or 2;
R² is selected from OH, C₁-C₄ alkyl-C(O)O—, alkylamino of 1 to 4 carbon atoms, dialkylamino of 2 to 8 carbons, aryl, cycloalkyl of 3 to 8 carbon atoms, C₁-C₉ heterocycloalkyl and (alkyl)₃Si—O— containing 3 to 12 carbon atoms, any of which may be substituted;
comprising the step of reacting a reagent of formula (II): 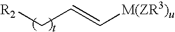
in the presence of a source of Pd(0) metal with a compound of formula (VII): 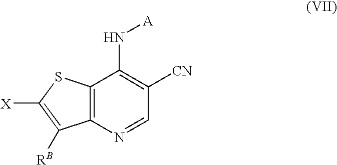
wherein:
X is selected from O-triflate, Br, I and Cl;
M is Sn or B;
Z is a bond or an oxygen atom, with the proviso that Z is a bond when M is Sn and Z is oxygen when M is B;
u is 1, 2 or 3; and
R³ is independently selected from H and alkyl of 1 to 12 carbon atoms, or two R³ groups taken together with Z and M can form a 3 to 8 membered heterocyclic ring; or salts thereof.

18. The method of claim 17, wherein A is phenyl, which may be substituted.

19. The method of claim 17, wherein R^{B} is H.

20. The method of claim 18, wherein A is substituted by H, Cl, OCH₃ or —S-heteroaryl.

21. The method of claim 17, wherein M is Sn and Z is a bond.

22. The method of claim 17, wherein M is B and Z is oxygen.

23. The method of claim 17, wherein the source of Pd(0) is Pd(PPh₃)₄.

24. The method of any of claims 1, 9 or 17 further comprising using a solvent selected from the group consisting from NMP, toluene, benzene, toluene/ethanol/water (10:1:1), DMF, THF, and DMF/THF (1:1)

25. The method of claim 24, wherein the solvent is toluene/ethanol/water (10:1:1).

26. The method of claim 24, wherein the solvent is NMP.

27. The method of any of claims 1, 9 or 17 further comprising the step of heating the reaction to a temperature in the range of about 80° C. to about 120° C.

28. The method of claim 27, wherein the temperature is at least 90° C.

29. The method of claim 27, wherein the temperature is at least 105° C.

30. The method of claim 1, wherein the compound is
4-[(2,4-Dichloro-5-methoxyphenyl)amino]-6-methoxy-7-[(1E)-4-(4-methyl piperazin-1-yl)but-1-enyl]quinoline-3-carbonitrile.

31. The method of claim 9, wherein the compound is
4-[(2,4-Dichloro-5-methoxyphenyl)amino]-6-methoxy-7-[(1E)-4-(4-methyl piperazin-1-yl)but-1-enyl]quinoline-3-carbonitrile.

32. The method of claim 1, wherein the compound is selected from the group consisting of
4-[(2,4-dichlorophenyl)amino]-7-[(1E)-5-morpholin-4-ylpent-1-enyl]quinoline-3-carbonitrile,
4-[(2,4-dichlorophenyl)amino]-7-[(1E)-6-morpholin-4-ylhex-1-enyl]quinoline-3-carbonitrile,
4-[(2,4-dichloro-5-methoxyphenyl)amino]-6-methoxy-7-[(1E)-5-morpholin-4-ylpent-1-enyl]quinoline-3-carbonitrile,
4-({3-chloro-4-[(1-methyl-1H-imidazol-2-yl)thio]phenyl}amino)-7-[(1E)-3-hydroxyprop-1-enyl]quinoline-3-carbonitrile,
4-({3-chloro-4-[(1-methyl-1H-imidazol-2-yl)thio]phenyl}amino)-7-[(1E)-4-hydroxybut-1-enyl]quinoline-3-carbonitrile,
(2E)-3-[4-({3-chloro-4-[(1-methyl-1H-imidazol-2-yl)thio]phenyl}amino)-3-cyanoquinolin-7-yl]prop-2-enyl acetate,
4-({3-chloro-4-[(1-methyl-1H-imidazol-2-yl)thio]phenyl}amino)-7-[(1E)-3-morpholin-4-ylprop-1-enyl]quinoline-3-carbonitrile,
4-({3-chloro-4-[(1-methyl-1H-imidazol-2-yl)thio]phenyl}amino)-7-[(1E)-4-(diethylamino)but-1-enyl]quinoline-3-carbonitrile,
4-({3-chloro-4-[(1-methyl-1H-imidazol-2-yl)thio]phenyl}amino)-7-[(1E)-4-(dimethylamino)but-1-enyl]quinoline-3-carbonitrile,
4-({3-chloro-4-[(1-methyl-1H-imidazol-2-yl)thio]phenyl}amino)-7-[(1E)-4-morpholin-4-ylbut-1-enyl]quinoline-3-carbonitrile,
4-({3-chloro-4-[(1-methyl-1H-imidazol-2-yl)thio]phenyl}amino)-7-[(1E)-3-(diethylamino)prop-1-enyl]quinoline-3-carbonitrile,
4-({3-chloro-4-[(1-methyl-1H-imidazol-2-yl)thio]phenyl}amino)-7-[(1E)-4-pyrrolidin-1-ylbut-1-enyl]quinoline-3-carbonitrile,
4-({3-chloro-4-[(1-methyl-1H-imidazol-2-yl)thio]phenyl}amino)-7-[(1E)-3-dimethylamino)prop-1-enyl]quinoline-3-carbonitrile,
4-({3-chloro-4-[(1-methyl-1H-imidazol-2-yl)thio]phenyl}amino)-7-[(1E)-4-piperidin-1-ylbut-1-enyl]quinoline-3-carbonitrile,
4-({3-chloro-4-[(1-methyl-1H-imidazol-2-yl)thio]phenyl}amino)-7-[(1E)-3-pyrrolidin-1-ylprop-1-enyl]quinoline-3-carbonitrile,
4-({3-chloro-4-[(1-methyl-1H-imidazol-2-yl)thio]phenyl}amino)-7-[(1E)-4-hydroxybut-1-enyl]-6-methoxyquinoline-3-carbonitrile,
4-({3-chloro-4-[(1-methyl-1H-imidazol-2-yl)thio]phenyl}amino)-7-[(1E)-5-pyrrolidin-1-ylpent-1-enyl]quinoline-3-carbonitrile,
4-({3-chloro-4-[(1-methyl-1H-imidazol-2-yl)thio]phenyl}amino)-7-[(1E)-4-(diethylamino)but-1-enyl]-6-methoxyquinoline-3-carbonitrile,
4-({3-chloro-4-[(1-methyl-1H-imidazol-2-yl)thio]phenyl}amino)-6-methoxy-7-[(1E)-4-pyrrolidin-1-ylbut-1-enyl]quinoline-3-carbonitrile,
4-({3-chloro-4-[(1-methyl-1H-imidazol-2-yl)thio]phenyl}amino)-7-[(1E)-4-(4-methylpiperazin-1-yl)but-1-enyl]quinoline-3-carbonitrile,
4-({3-chloro-4-[(1-methyl-1H-imidazol-2-yl)thio]phenyl}amino)-6-methoxy-7-[(1E)-4-(4-methylpiperazin-1-yl)but-1-enyl]quinoline-3-carbonitrile,
4-({3-chloro-4-[(1-methyl-1H-imidazol-2-yl)thio]phenyl}amino)-7-[(1E)-4-(dimethylamino)but-1-enyl]-6-methoxyquinoline-3-carbonitrile,
4-[(2,4-dichloro-5-methoxyphenyl)amino]-7-[(1E)-4-morpholin-4-ylbut-1-enyl]quinoline-3-carbonitrile,
4-[(2,4-dichloro-5-methoxyphenyl)amino]-6-methoxy-7-[(1E)-4-morpholin-4-ylbut-1-enyl]quinoline-3-carbonitrile,
4-({3-chloro-4-[(1-methyl-1H-imidazol-2-yl)thio]phenyl}amino)-7-[(1E)-5-(diethylamino)pent-1-enyl]quinoline-3-carbonitrile,
4-({3-chloro-4-[(1-methyl-1H-imidazol-2-yl)thio]phenyl}amino)-7-[(1E)-5-(diethylamino)pent-1-enyl]-6-methoxyquinoline-3-carbonitrile,
4-({3-chloro-4-[(1-methyl-1H-imidazol-2-yl)thio]phenyl}amino)-7-[(1E)-5-(4-methylpiperazin-1-yl)pent-1-enyl]quinoline-3-carbonitrile,
4-({3-chloro-4-[(1-methyl-1H-imidazol-2-yl)thio]phenyl}amino)-6-methoxy-7-[(1E)-5-(4-methylpiperazin-1-yl)pent-1-enyl]quinoline-3-carbonitrile,
4-[(2,4-dichloro-5-methoxyphenyl)amino]-6-methoxy-7-[(1E)-4-(4-methylpiperazin-1-yl)but-1-enyl]quinoline-3-carbonitrile,
4-[(2,4-dichloro-5-methoxyphenyl)amino]-7-[(1E)-4-hydroxybut-1-enyl]-6-methoxyquinoline-3-carbonitrile,
7-[(1E)-4-morpholin-4-ylbut-1-enyl]-4-[(3,4,5-trimethoxyphenyl)amino]quinoline-3-carbonitrile,
4-[(2,4-dichloro-5-methoxyphenyl)amino]-6-methoxy-7-[(1E)-3-morpholin-4-ylprop-1-enyl]quinoline-3-carbonitrile,
6-methoxy-7-[(1E)-4-(4-methylpiperazin-1-yl)but-1-en-1-yl]-4-[(3,4,5-trimethoxyphenyl)amino]quinoline-3-carbonitrile,
4-[(2,4-dichloro-5-methoxyphenyl)amino]-6-methoxy-7-[(1E)-4-piperidin-1-ylbut-1-enyl]quinoline-3-carbonitrile,
4-[(2,4-dichlorophenyl)amino]-7-[(1E)-4-morpholin-4-ylbut-1-enyl]quinoline-3-carbonitrile,
4-[(2,4-dichlorophenyl)amino]-7-[(1E)-3-morpholin-4-ylprop-1-enyl]quinoline-3-carbonitrile,
4-({3-chloro-4-[(1-methyl-1H-imidazol-2-yl)thio]phenyl}amino)-7-[(1E)-3-(4-methylpiperazin-1-yl)prop-1-enyl]quinoline-3-carbonitrile,
4-[(2,4-dichloro-5-methoxyphenyl)amino]-6-methoxy-7-[(1E)-6-morpholin-4-ylhex-1-enyl]quinoline-3-carbonitrile,
4-[(2,4-dichlorophenyl)amino]-7-[(1E)-11-morpholin-4-ylundec-1-enyl]quinoline-3-carbonitrile,
4-[(2,4-dichloro-5-methoxyphenyl)amino]-6-methoxy-7-[(1E)-11-morpholin-4-ylundec-1-enyl]quinoline-3-carbonitrile,
4-({3-chloro-4-[(1-methyl-1H-imidazol-2-yl)thio]phenyl}amino)-7-[(1E)-3-piperidin-1-ylprop-1-enyl]quinoline-3-carbonitrile,
4-({3-chloro-4-[(1-methyl-1H-imidazol-2-yl)thio]phenyl}amino)-7-[(1E)-3-(4-pyrrolidin-1-ylpiperidin-1-yl)prop-1-enyl]quinoline-3-carbonitrile,
4-({3-chloro-4-[(1-methyl-1H-imidazol-2-yl)thio]phenyl}amino)-7-[(1E)-3-(4-ethyl piperazin-1-yl)prop-1-enyl]quinoline-3-carbonitrile,
4-({3-chloro-4-[(1-methyl-1H-imidazol-2-yl)thio]phenyl}amino)-7-[(1E)-4-(4-ethylpiperazin-1-yl)but-1-enyl]quinoline-3-carbonitrile,
4-({3-chloro-4-[(1-methyl-1H-imidazol-2-yl)thio]phenyl}amino)-7-[(1E)-4-(tetrahydro-2H-pyran-2-yloxy)but-1-enyl]quinoline-3-carbonitrile,
7-((1E)-4-{[tert-butyl(dimethyl)silyl]oxy}but-1-enyl)-4-[(2,4-dichloro-5-methoxyphenyl)amino]-6-methoxyquinoline-3-carbonitrile,
4-({3-chloro-4-[(1-methyl-1H-imidazol-2-yl)thio]phenyl}amino)-7-[(1E)-4-(4-pyrrolidin-1-ylpiperidin-1-yl)but-1-enyl]quinoline-3-carbonitrile,
4-[(2,4-dichloro-5-methoxyphenyl)amino]-7-[(1E)-4-(4-methylpiperazin-1-yl)but-1-enyl]quinoline-3-carbonitrile,
4-({3-chloro-4-[(1-methyl-1H-imidazol-2-yl)thio]phenyl}amino)-6-fluoro-7-[(1E)-4-(4-methylpiperazin-1-yl)but-1-enyl]quinoline-3-carbonitrile,
4-[(2,4-dichloro-5-methoxyphenyl)amino]-7-[(1E)-4-(dimethylamino)but-1-enyl]-6-methoxyquinoline-3-carbonitrile,
4-[(2,4-dichloro-5-methoxyphenyl)amino]-7-[(1E)-4-(4-ethylpiperazin-1-yl)but-1-enyl]-6-methoxyquinoline-3-carbonitrile,
7-[(1E)-4-hydroxybut-1-en-1-yl]-6-methoxy-4-[(3,4,5-trimethoxyphenyl)amino]quinoline-3-carbonitrile,
4-[(2,4-dichloro-5-methoxyphenyl)amino]-6-methoxy-7-[(1E)-4-pyrrolidin-1-ylbut-1-en-1-yl]quinoline-3-carbonitrile,
4-[(2,4-dichloro-5-methoxyphenyl)amino]-8-[(1E)-4-morpholin-4-ylbut-1-enyl]quinoline-3-carbonitrile,
4-[(2,4-dichlorophenyl)amino]-6-[(1E)-4-morpholin-4-ylbut-1-enyl]quinoline-3-carbonitrile,
4-[(2,4-dichloro-5-methoxyphenyl)amino]-6-[(1E)-4-morpholin-4-ylbut-1-enyl]quinoline-3-carbonitrile,
4-[(2,4-dichlorophenyl)amino]-6-[(1E)-4-(4-methylpiperazin-1-yl)but-1-enyl]quinoline-3-carbonitrile, and
6-[(1E)-4-morpholin-4-ylbut-1-enyl]-4-[(3,4,5-trimethoxyphenyl)amino]quinoline-3-carbonitrile.

33. The method of claim 17, wherein the compound is selected from the group consisting of
7-({3-chloro-4-[(1-methyl-1H-imidazol-2-yl)thio]phenyl}amino)-2-[(1E)-4-pyrrolidin-1-ylbut-1-enyl]thieno[3,2-b]pyridine-6-carbonitrile,
7-({3-chloro-4-[(1-methyl-1H-imidazol-2-yl)thio]phenyl}amino)-2-[(1E)-4-(4-pyrrolidin-1-ylpiperidin-1-yl)but-1-enyl]thieno[3,2-b]pyridine-6-carbonitrile,
7-({3-chloro-4-[(1-methyl-1H-imidazol-2-yl)thio]phenyl}amino)-2-[(1E)-4-(4-ethylpiperazin-1-yl)but-1-enyl]thieno[3,2-b]pyridine-6-carbonitrile,
7-({3-chloro-4-[(1-methyl-1H-imidazol-2-yl)thio]phenyl}amino)-2-[(1E)-4-(4-methylpiperazin-1-yl)but-1-en-1-yl]thieno[3,2-b]pyridine-6-carbonitrile, and
7-({3-chloro-4-[(1-methyl-1H-imidazol-2-yl)thio]phenyl}amino)-2-[(1E)-3-(dimethylamino)prop-1-en-1-yl]thieno[3,2-b]pyridine-6-carbonitrile.

34. A compound of formula:
4-[(2,4-Dichloro-5-methoxyphenyl)amino]-6-methoxy-7-[(1E)-4-(4-methyl piperazin-1-yl)but-1-enyl]quinoline-3-carbonitrile or pharmaceutically acceptable salts thereof.
